(19) United States
(12) Patent Application Publication
(10) Pub. No.: US 2026/0165776 A1
(43) Pub. Date: Jun. 18, 2026

(54) NEUROMODULATION THERAPY INCLUDING MONOPOLAR AND BIPOLAR ENERGY DELIVERY

(71) Applicant: Medtronic Ireland Manufacturing Unlimited Company, Dublin (IE)

(72) Inventors: Douglas A. Hettrick, Andover, MN (US); Stefan S. Tunev, Santa Rosa, CA (US); Carlos F. Haro, New York, NY (US)

(21) Appl. No.: 19/125,048

(22) PCT Filed: Oct. 9, 2023
(86) PCT No.: PCT/EP2023/077904

§ 371 Date: Apr. 28, 2025

Related U.S. Application Data
(60) Provisional application No. 63/381,438, filed on Oct. 28, 2022.

(51) Int. Cl. A61B 18/14; A61B 18/00; A61B 18/12

(52) U.S. Cl. CPC A61B 18/1492; A61B 18/1206; A61B 2018/00791; A61B 2018/00875; A61B 2018/1253; A61B 2018/1467

(57) ABSTRACT

In some examples, a medical device system includes signal generation circuitry configured to deliver energy to a target tissue site within a patient via one or more electrodes of a plurality of electrodes disposed along an elongated body of a catheter, sensing circuitry configured to sense a parameter at or near the target tissue site, and control circuitry. The control circuitry is configured to cause the signal generation circuitry to deliver monopolar energy to the target tissue site via one or more electrodes of the plurality of electrodes, determine that the sensed parameter satisfies a threshold condition, and based on determining that the sensed parameter satisfies the threshold condition, cause the signal generation circuitry to deliver bipolar energy to the target tissue site via two or more electrodes of the plurality of electrodes.

### TECHNICAL FIELD

The present technology is related to neuromodulation therapy.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

### SUMMARY

The present disclosure describes devices, systems, and methods for neuromodulation, such as renal neuromodulation. In examples described herein, control circuitry of a medical device system is configured to control delivery of monopolar neuromodulation energy and bipolar neuromodulation energy based on a sensed parameter. In some examples, the control circuitry is configured to cause the signal generation circuitry to deliver monopolar energy to the target tissue site and, based on determining that a sensed parameter satisfies a threshold condition, cause the signal generation circuitry to deliver bipolar energy to the target tissue site. For example, based on determining that the sensed parameter satisfies the threshold condition, the control circuitry may cause the signal generation circuitry to terminate delivery of the monopolar energy and begin delivery of the bipolar energy to the target tissue site.

Compared to bipolar energy, monopolar energy may penetrate deeper into a tissue of a patient relative to the neuromodulation catheter, e.g., because the monopolar energy is delivered between one or more electrodes of the neuromodulation catheter and a reference electrode (e.g., on the outside of the body of a patient), rather than between electrodes of the catheter (e.g., on the same catheter) as in the case of bipolar energy delivery. Thus, depending on the particular target tissue site, tissue conditions, or the like, the control circuitry may select one of monopolar energy or bipolar energy to better target the target tissue (e.g., nerves) and minimize adverse effects of the energy delivery on non-target tissue.

The medical device system includes sensing circuitry configured to sense one or more parameters (e.g., temperature, impedance or the like) within the patient, e.g., via sensors on the neuromodulation catheter or separate from the neuromodulation catheter. In some locations within the vasculature of the patient, a target nerve may be closer to or further away from a blood vessel. The sensed parameters can be indicative of a distance between the target nerve and the blood vessel (e.g., by indicating a diameter of the blood vessel or the like). During delivery of neuromodulation therapy, control circuitry of the medical device system may determine the effects of neuromodulation therapy based on the one or more sensed parameters. The control circuitry may determine whether the effects of the therapy are within an acceptable range (e.g., to prevent unintended effects at the target tissue site) by determining whether the one or more sensed parameters satisfy one or more threshold conditions defining the acceptable range of effects of the therapy.

The devices, systems, and methods described in this disclosure may provide one or more benefits over other neuromodulation devices, systems, and methods. Switching between monopolar energy delivery and bipolar energy delivery can reduce adverse impacts to tissue at or near the target tissue site while maintaining efficacious neuromodulation energy delivery. For example, switching from monopolar energy delivery to bipolar energy delivery can help reduce the extent of the energy penetration into surrounding tissue, which can reduce heating of the non-target tissue. Further, switching from delivery of monopolar energy to bipolar energy based on determining that the one or more sensed parameters satisfy at least one threshold condition may allow for continued neuromodulation of the target nerve(s) while still reducing energy delivery to non-target tissue.

In some examples, the disclosure describes a medical device system comprising: signal generation circuitry configured to delivery energy to a target tissue site within a patient via one or more electrodes of a plurality of electrodes disposed along an elongated body of a catheter; sensing circuitry configured to sense a parameter at or near the target tissue site; and control circuitry configured to: cause the signal generation circuitry to deliver monopolar energy to the target tissue site via one or more electrodes of the plurality of electrodes; determine that the sensing parameter satisfies a threshold condition; and based on determining that the sensed parameter satisfies the threshold condition, cause the signal generation circuitry to delivery bipolar energy to the target tissue site via two or more electrodes of the plurality of electrodes.

In some examples, the disclosure describes a method comprising: controlling, by control circuitry, signal generation circuitry to deliver monopolar energy via one or more electrodes of a plurality of electrodes of a catheter; receiving, by the control circuitry and from sensing circuitry, a signal indicative of a parameter at or near the target tissue site; determining, by the control circuitry, that the sensed parameter satisfies a threshold condition; and based on determining that the sensed parameter satisfies the threshold condition, controlling, by the control circuitry, the signal generation circuitry to deliver a bipolar energy to the target tissue site via two or more electrodes of the plurality of electrodes.

In some examples, the disclosure describes A medical device comprising signal generation circuitry configured to deliver energy to tissue of a patient via a plurality of electrodes of a catheter; sensing circuitry configured to sense a parameter within the patient; and control circuitry configured to: cause signal generation circuitry to deliver monopolar energy to a first target tissue site within a blood vessel of the patient via one or more electrodes of the plurality of electrodes; determine, based on the parameter, a size of the blood vessel at a second target tissue site within the blood vessel or a distance between a nerve and the blood vessel at the second target tissue site; and based on the determined size of the blood vessel or the determined distance, cause the signal generation circuitry to deliver bipolar energy to the second target tissue site via two or more electrodes of the plurality of electrodes.

Further disclosed herein is a medical device system that includes signal generation circuitry configured to deliver energy to a target tissue site within a patient via one or more electrodes of a plurality of electrodes disposed along an elongated body of a catheter, sensing circuitry configured to sense a parameter at or near the target tissue site, and control circuitry, wherein the control circuitry is configured to cause the signal generation circuitry to deliver monopolar energy to the target tissue site via one or more electrodes of the plurality of electrodes, determine that the sensed parameter satisfies a threshold condition, and based on determining that the sensed parameter satisfies the threshold condition, cause the signal generation circuitry to deliver bipolar energy to the target tissue site via two or more electrodes of the plurality of electrodes.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example neuromodulation system.

FIG. 2 is a block diagram illustrating an example control device of FIG. 1.

FIG. 3 is a conceptual diagram illustrating a distal portion of an elongated member of the catheter of FIG. 1 positioned within a blood vessel.

FIG. 4A is a conceptual diagram illustrating an example delivery of monopolar energy via the catheter of FIG. 3.

FIG. 4B is a conceptual diagram illustrating an example delivery of multipolar energy via the catheter of FIG. 3.

FIG. 5 is a conceptual diagram illustrating example energy delivery profiles of different energy modes of the neuromodulation system of FIG. 1.

FIG. 6 is a flow diagram illustrating an example method of switching from delivery of monopolar energy to delivery of bipolar energy based on a position of the catheter of FIG. 1 within a patient.

FIG. 7 is a flow diagram illustrating an example method of switching from delivery of monopolar energy to delivery of bipolar energy based on a satisfaction of a threshold condition.

FIG. 8 illustrates an example technique for accessing a renal artery and modulating renal nerves with the system of FIG. 1 in accordance with some examples of the present disclosure.

FIG. 9 is a conceptual illustration of an example sympathetic nervous system (SNS) illustrating how the brain communicated with the body via the SNS.

FIG. 10 is an enlarged anatomic view of nerves innervating a left kidney to form the renal plexus surrounding the left renal artery.

FIG. 11 is an anatomic view of a human body depicting neural efferent and afferent communication between the brain and kidneys.

FIG. 12 is a conceptual view of a human body depicting neural efferent and afferent communication between the brain and kidneys.

FIG. 13 is an anatomic view of the arterial vasculature of a human.

FIG. 14 is an anatomic view of the venous vasculature of a human.

### DETAILED DESCRIPTION

The present disclosure describes devices, systems, and method for neuromodulation, such as renal neuromodulation, using monopolar and bipolar energy delivery (e.g., radiofrequency (RF) energy delivery). Although renal neuromodulation is primarily described herein, devices, systems, and techniques described herein may be applied to other types of neuromodulation, such as neuromodulation performed on nerves other than the renal nerves, at sites other than within a renal vessel, or both. In general, the devices, systems, and techniques described herein may be used to perform neuromodulation from within any suitable anatomical lumen that has nerves adjacent to the anatomical lumen. Example anatomical lumens include the celiac trunk and its branches (including the common hepatic artery and its branches (including the gastroduodenal artery and its branches, the right gastric artery and its branches, and the proper hepatic artery and its branches), the left gastric artery and its branches, and the splenic artery and its branches), the superior mesenteric artery and its branches, the gonadal artery and its branches, the inferior mesenteric artery and its branches, and the like. Further, although neuromodulation from within one or more veins, such as a renal vein and its branches, a hepatic vein and its branches, an intercostal vein and its branches, or the like. In some implementations the devices, systems, and techniques described herein may be used to perform neuromodulation from within two or more anatomical lumens, e.g., in the renal arteries and the common hepatic artery, or any other combination of two or more anatomical lumens. In addition, the systems, devices, and methods described herein may be useful for neuromodulation within a body lumen other than a vessel, for extravascular neuromodulation and/or for use in therapies other than neuromodulation.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device.

Neuromodulation, such as renal denervation, may be accomplished using one or more of a variety of treatment modalities, including RF energy, microwave energy, ultrasound energy, heat, cryogenic cooling, a chemical agent, or the like. To perform intravascular neuromodulation, a neuromodulation catheter may be delivered to a blood vessel, such as a renal artery, of a patient. In some examples, the neuromodulation catheter includes one or more expandable members, each of which includes one or more electrodes. The one or more expandable members can include, for example, a shape memory element configured to assume an expanded shape (e.g., a helical, spiral, basket, or stent-like shape), a balloon, or the like. Each expandable member is configured to position one or more electrodes in apposition to the vessel wall to transfer energy (e.g., RF energy) to or from tissue surrounding the vessel wall.

In some examples, a neuromodulation system is configured to deliver energy to the tissue surrounding the vessel wall in the form of monopolar energy and/or multipolar energy (e.g., primarily referred to bipolar energy herein). When the neuromodulation system is delivering monopolar energy to the tissue, the neuromodulation system may deliver energy from one or more cathode electrodes disposed on an elongated body of a catheter disposed within the blood vessel to an anode electrode (e.g., a reference electrode) separate from the catheter. The reference electrode can be, for example, a ground pad disposed on a surface of a patient (e.g., on the skin of the patient) or in a location spaced from the catheter within the patient.

In some examples, the energy is delivered as bipolar energy, in which the energy travels from one or more cathode electrodes disposed on the catheter to one or more anode electrodes disposed on the catheter. Because the monopolar energy is delivered between one or more electrodes of the neuromodulation catheter and the reference electrode and/or because the monopolar energy may be delivered via a relatively higher amplitude electrical signal than bipolar energy, the monopolar energy may penetrate deeper into tissue compared to bipolar energy, which can lead to increased heating of non-target tissue, which may lead to adverse impacts to the tissue.

In some locations within the body of the patient, a target nerve(s) for the neuromodulation therapy may be closer to or further away from a blood vessel containing the neuromodulation catheter. In such examples, a clinician may need to apply less or more energy to the target nerve(s), respectively, to successfully complete the neuromodulation therapy. In examples described herein, the clinician may switch between monopolar and bipolar energies to adjust the amount of energy delivered to the target nerve(s), e.g., based on a distance between the blood vessel and the target nerve(s).

In some examples described herein, the medical system is configured to sense one or more parameters at or near the target tissue site (e.g., via one or more electrodes of the catheter or via a separate sensor) and select between monopolar energy delivery and bipolar energy delivery based on the sensed one or more parameters. The one or more parameters may include, but are not limited to, a temperature sensed near the plurality of electrodes, a change in the sensed temperature, an impedance of one or more electrodes, or a change in impedance of the one or more electrodes. Based on determining that the one or more sensed parameters satisfy at least one threshold condition (e.g., a threshold temperature, a threshold rate of change in temperature, a threshold impedance value, a threshold rate of change in the impedance value), control circuitry of the medical system can control the medical system to terminate delivery of monopolar energy to the tissue of the patient and, in some examples, switch to bipolar energy delivery.

In some examples, a medical system is configured to deliver energy to a target tissue site via one or more electrodes located within a blood vessel to ablate at least some nerves adjacent to the blood vessel. The nerve locations with respect to the vessel may vary widely among patients, particularly for renal nerves around a renal vessel. For example, a distance between the renal nerve and a vessel wall of the renal vessel may be greater than a distance between the renal nerve and a vessel wall of one or more branch vessels connected to the renal vessel at a bifurcation.

In accordance with some examples of this disclosure, a neuromodulation system includes control circuitry configured to switch between different modes of therapy delivery (e.g., between monopolar energy delivery and/or bipolar energy delivery). In some examples, the control circuitry is configured to switch between monopolar energy delivery and bipolar energy delivery based on a distance between target nerve(s) and a blood vessel in which a catheter including the electrodes is disposed. The distance can be determined using any suitable technique, such as based on a sensed parameter that changes as a function of and is indicative of the distance, or based on medical imaging and/or user input.

By switching between monopolar energy and bipolar energy, the neuromodulation system described in this disclosure may treat the target nerve(s) while minimizing overpenetration of the tissue of the patient by the delivered energy. In some examples, the control circuitry is configured to switch from monopolar energy delivery to bipolar energy delivery based on a determination that a sensed parameter satisfies a threshold condition. Switching to bipolar energy delivery may allow the neuromodulation system to continue delivering therapeutic energy to the target nerve(s) without causing unintended results (e.g., by the continued delivery of monopolar energy).

While RF energy delivered as an electrical signal is primarily referred to herein, in other examples, the description here can apply to other types of energy, such as, but not limited to, microwave energy or variations of the energy application such as gating or pulsing.

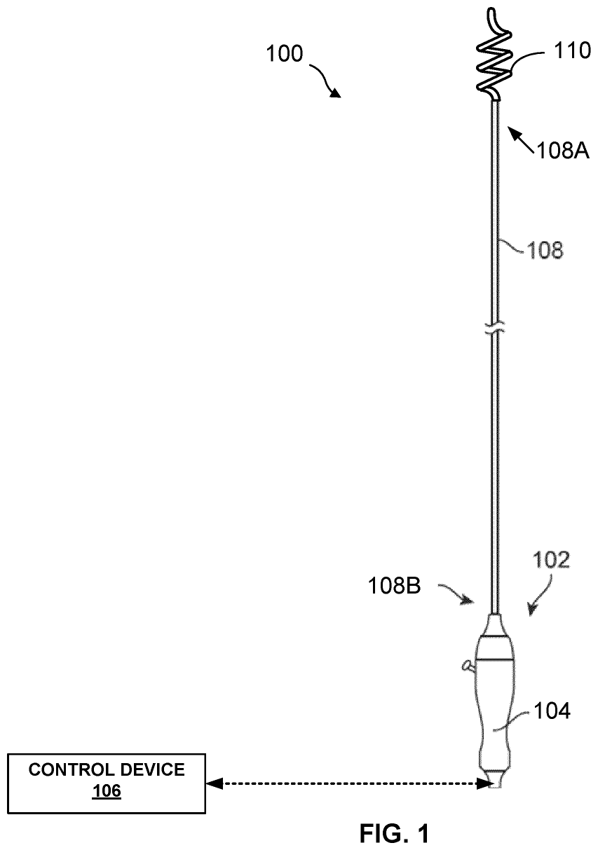

FIG. 1 is a partially schematic illustration of an example neuromodulation system 100 (“system 100”) configured to switch from monopolar energy delivery to bipolar energy delivery, e.g., based on a sensed parameter. The sensed parameter can be, for example, indicative of the effects of the delivery neuromodulation therapy (e.g., tissue impedance or temperature) and/or can be indicative of a distance between the vessel wall and a target nerve (e.g., can be indicative of a diameter of the blood vessel in which a catheter is positioned). As shown in FIG. 1, system 100 includes a neuromodulation catheter 102, which includes a handle 104, control device 106, and an elongated member 108 attached to handle 104. Elongated member 108 includes a distal portion 108A and a proximal portion 108b. Distal portion 108A includes a therapy delivery element 110.

In the example shown in FIG. 1, therapy delivery element 110 includes a plurality of electrodes disposed along elongated member 108. Therapy delivery element 110 may be electrically connected to control device 106 via a plurality of electrical conductors disposed within elongated member 108 and/or handle 104. Control device 106 may, via the plurality of electrodes, deliver therapeutic energy (e.g., RF energy) to and/or sense one or more parameters of tissue of a vessel wall of a blood vessel of the patient. Although FIG. 1 illustrates catheter 102 as having one therapy delivery element 110, other example catheters may include two or more therapy delivery elements 110.

Elongated member 108 may have any suitable outer diameter, and the diameter can be constant along the length of elongated member 108 or may vary along the length of elongated member 108. In some examples, elongated member 108 can be 2, 3, 4, 5, 6, or 7 French or another suitable size.

Distal portion 108A of elongated member 108 is configured to be advanced within an anatomical lumen of a human patient to locate therapy delivery element 110 at a target tissue site within or otherwise proximate to the anatomical lumen. For example, elongated member 108 may be configured to position therapy delivery element 110 within a blood vessel, a ureter, a duct, an airway, or another naturally occurring lumen within the human body. The examples described herein focus on the anatomical lumen being a blood vessel, such as a renal vessel, but it will be understood that similar techniques may be used with other anatomical lumens. In certain examples, intravascular delivery of therapy delivery element 110 includes percutaneously inserting a guidewire (not shown in FIG. 1) into a vessel of a patient and moving elongated member 108 and/or therapy delivery element 110 along the guidewire until therapy delivery element 110 reach a target tissue site (e.g., a renal artery). For example, distal portion 108A of elongated member 108 may define a passageway for engaging the guidewire for delivery of therapy delivery element 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter or sheath (not shown in FIG. 1), or other guide device.

Once at the target tissue site, therapy delivery element 110 can be configured to deliver therapy, such as RF energy to provide or facilitate neuromodulation therapy at the target tissue site. For ease of description, the following discussion will be primarily focused on delivering RF energy, in which example therapy delivery element 110 includes a plurality of electrodes. Therapy delivery element 110 may deliver RF energy to the tissue of the patient by delivering monopolar energy and/or bipolar energy (e.g., in the form of a monopolar or a bipolar electrical signal) to the tissue. It will be understood, however, that in other examples, therapy delivery element 110 may include elements or structures configured to deliver other types of therapy in addition to or instead of RF energy.

In the example shown in FIG. 1, therapy delivery element 110 is configured to assume a delivery configuration in which therapy delivery element 110 defines a relatively smaller radial extent (a relatively low profile), and a radially expanded configuration in which therapy delivery element 110 defines a relatively larger radial extent. Distal portion 108A may be delivered through vasculature of the patient to the target tissue site while therapy delivery element 110 is in the delivery configuration. In some examples, in the radially expanded configuration, therapy delivery element 110 defines a helical, a spiral, a loop, a basket, or a stent-like configuration. In the radially expanded configuration, therapy delivery element 110 is configured to position one or more electrodes of the plurality of electrodes carried by therapy delivery element 110 near a vessel wall, e.g., in apposition to the vessel wall. In other examples, therapy delivery element includes a balloon configured to expand from a relatively low profile delivery configuration to an expanded configuration in order to position one or more electrodes of the plurality of electrodes carried by therapy delivery element 110 near a vessel wall.

The plurality of electrodes may be positioned along therapy delivery element 110 such that, when therapy delivery element 110 is in the radially expanded configuration, the electrodes are spaced around an inner perimeter (e.g., circumference) of the vessel wall. In some examples, the electrodes may be positioned along therapy delivery element 110 such that, when therapy delivery element 110 is in the radially expanded configuration, the electrodes are substantially evenly spaced around an inner perimeter (e.g., circumference) of the vessel wall.

Control device 106 includes signal generation circuitry configured to generate and deliver RF energy to the patient via the one or more electrodes disposed on therapy delivery element 110. Control device 106 further includes sensing circuitry configured to sense one or more parameters (e.g., temperature, impedance, or the like) from the patient at or near a target tissue site via the one or more electrodes and/or sensors and adjust the RF energy (e.g., between monopolar energy and bipolar energy) based on the sensed parameters. For example, control device 106 can be configured to switch from monopolar energy delivery to bipolar energy to the target tissue site in response to a determination that the sensed parameters satisfy one or more threshold conditions and/or that a distance between the target nerve(s) and the blood vessel satisfies a threshold condition.

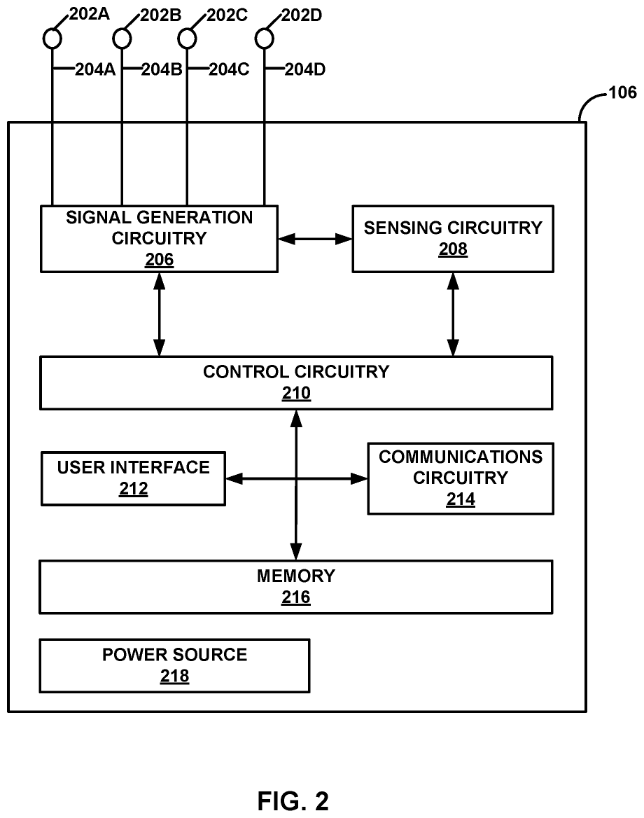

FIG. 2 is a block diagram illustrating an example control device 106 of FIG. 1. In this example, control device 106 includes signal generation circuitry 206, sensing circuitry 208, control circuitry 210, user interface 212, communications circuitry 214, memory 216, and a power source 218 that provides operational power to the other components. Control device 106 is electrically connected to electrodes 202A-202D (also referred to as “electrodes 202”) disposed on therapy delivery element 110 of catheter 102 (not pictured) via electrical conductors 204A-204D (also referred to as “electrical conductors 204”). In the example shown in FIG. 2, each electrode 202A-202D is electrically coupled to a separate electrical conductor 204A-204D such that each electrode is independently and separately activatable. The various circuitry may be, or include, programmable or fixed function circuitry configured to perform the functions attributed to the respective circuitry.

Memory 216 may store computer-readable instructions that, when executed by control circuitry 210, cause control circuitry 210 and control device 106 to perform various functions described herein. Memory 216 may be a storage device or other non-transitory medium. Memory 216 may include any volatile, non-volatile, magnetic, optical, or electrical media, such as random-access memory (RAM), read-only memory (ROM), non-volatile RAM (NVRAM), electrically-erasable programmable ROM (EEPROM), flash memory, or any other digital media.

Control circuitry 210 (alternatively referred to as “processing circuitry 210”) may include any one or more of a microprocessor, a controller, a digital signal processor (DSP), an application-specific integrated circuitry (ASIC), a field-programmable gate array (FPGA), discrete logic circuitry, or any other circuitry configured to provide the functions attributed to control circuitry 210 herein and may be embodied as firmware, hardware, software, or any combination thereof.

Electrodes 202 are electrically connected to signal generation circuitry 206 of control device 106 through electrical conductors 204. Electrical conductors 204 may have any suitable configuration, e.g., electrical wires, feedthrough assemblies, or the like extending inside handle 104 and elongated member 108 to therapy delivery element 110.

In some examples, as illustrated in FIG. 2, each of electrodes 202 is separately electrically connected to signal generation circuitry 206 via a corresponding electrical conductor of electrical conductors 204. In other examples, electrodes 202 may be electrically connected, via electrical conductors 204, to a switching circuitry configured to selectively couple signal generation circuitry 206 and/or sensing circuitry 208 to selected combinations of electrodes 202.

Each of electrodes 202 may be configured to function as a cathode electrode only, as an anode electrode only, or as both a cathode electrode and an anode electrode. In some examples, each of electrodes 202 may be configured to deliver monopolar energy only, to deliver bipolar energy only, or to deliver both monopolar energy and bipolar energy.

Signal generation circuitry 206 is configured to generate and delivery energy, e.g., in the form of an electrical signal, to the target tissue in the form of monopolar energy and bipolar energy. Although RF energy and signals are primarily referred to herein, in other examples, the energy can be other types of energy and signals, such as, but not limited to, microwave energy. Signal generation circuitry 206 is configured to deliver the generated RF signals to the target tissue site through one or more selected electrodes 202 and respective electrical conductors 204. Signal generation circuitry 206 has circuitry that enables it to deliver only monopolar energy, only bipolar energy (e.g., only bipolar energy), alternate between delivering monopolar energy and bipolar energy, or simultaneously deliver monopolar energy and bipolar energy to the target tissue site. Signal generation circuitry 206 may include, as examples, current or voltage sources, capacitors, charge pumps, or other signal generation circuitry. In some examples, signal generation circuitry 206 may receive sensed electrical signals from one or more of electrodes 202. Signal generation circuitry 206 may transmit the sensed electrical signals to sensing circuitry 208 and/or control circuitry 210.

Sensing circuitry 208 is configured to sense one or more parameters, such as temperature and/or impedance. For example, sensing circuitry 208 can be configured to sense the one or more parameters at or near the target tissue site. Sensing circuitry 208 has any suitable configuration and may, for example, include filters, amplifiers, analog-to-digital converters, or other circuitry configured to sense electrical signals via electrodes 202 or to convert the sensed electrical signals to one or more parameters (e.g., temperature/change in temperature of tissue near selected electrodes of electrodes 202, impedance/change in impedance sensed by the selected electrodes 202). In some examples, sensing circuitry 208 includes one or more thermocouples (e.g., connected to each of electrodes 202 via a pair of corresponding wires, which can, but need not, include conductors 204). In these examples, sensing circuitry 208 provides signals based on which control circuitry 210 can determine the temperature and/or change in temperature of tissue near the selected electrodes of electrodes 202 via the one or more thermocouples.

As discussed in further detail below, in some examples, control circuitry 210 is configured to control signal generation circuitry 206 to deliver neuromodulation to tissue of a patient via monopolar energy or bipolar energy based on one or more parameters sensed by sensing circuitry 208. The one or more sensed parameters can change as a function of the neuromodulation therapy delivery to tissue of a patient by system 100. For example, control circuitry 210 may determine values for the one or more parameters (e.g., temperature and/or impedance) and may compare the determined values against one or more threshold conditions to determine whether control device 106 should deliver monopolar energy or bipolar energy to the target tissue site or to cease delivery of RF energy to the target tissue site. The threshold conditions described herein, which can include, threshold values, threshold ranges or the like, can be stored, for example, in memory 216 of control device 106 or a memory of another device. In other examples, control circuitry 210 is configured to control signal generation circuitry 206 to deliver neuromodulation to the tissue of the patient via monopolar energy or bipolar energy based on user input, e.g., from a clinician.

As another example, control circuitry 210 may determine, based on the one or more parameters sensed via electrodes 202, a distance of target nerve(s) to a vessel wall at the target tissue site. In some examples, control circuitry 210 may compare the values for the sensed parameters against a threshold condition (e.g., a maximum threshold distance, a minimum threshold distance) to determine whether to deliver monopolar energy or bipolar energy to the target tissue site. In some examples, control circuitry 210 determines a vessel diameter based on a sensed parameter and correlates the vessel diameter to the distance of target nerve(s) to the vessel wall, and compares the vessel diameter to a threshold condition (e.g., a threshold vessel diameter) to determine whether to deliver monopolar energy or bipolar energy to the target tissue site. In some examples, control circuitry 210 may apply one or more rules or algorithms that associate different values for the distance of the target nerve(s) to the vessel wall with delivery of monopolar energy or bipolar energy and select one of the monopolar energy or bipolar energy based on the results. Control circuitry 210 may store these rules, algorithms, correlations, sensed parameters, the determined distances, and other information needed to carry out the therapies described herein in memory 216 and/or a memory of another device.

As discussed above, because the monopolar energy is delivered between one or more electrodes of the neuromodulation catheter and the reference electrode and/or because the monopolar energy may be delivered via a relatively higher amplitude electrical signal than bipolar energy, the monopolar energy may penetrate deeper into tissue (relative to the delivering electrode(s)) compared to bipolar energy, which can lead to increased heating of non-target tissue. The increased heating of non-target tissue may lead to adverse impacts to the tissue. The monopolar energy may lead to an increased rate of heating and/or magnitude of heating of non-target tissue relative to bipolar energy.

In some examples, control circuitry 210 is configured to determine values for the one or more parameters based on sensed data (e.g., generated by sensing circuitry 208) and determine, for each of the one or more parameters, whether the determined values satisfy a first threshold condition. The sensed one or more parameters may correspond to conditions of tissue of the patient at or near the target tissue site and can change as a function of the delivery of RF energy. The first threshold condition can include, for example, a first threshold temperature value, a first threshold rate of change/amount of change in temperature over time, a first threshold impedance value, a first threshold rate of change/amount of change in impedance over time, a first threshold difference between the determined temperature and a first predetermined temperature value (e.g., an average/mean temperature), or the like. The first threshold condition may define a range of acceptable effects of the RF energy on the target tissue site. Based on the determination that the determined one or more parameter values satisfy the first threshold condition, control circuitry 210 may cause (e.g., such as by directly or indirectly controlling) signal generation circuitry 206 to switch from delivering monopolar energy to the target tissue site to delivering bipolar energy to the target tissue site.

Because bipolar energy may cause a reduced rate of heating and/or a reduced magnitude of heating of non-target tissue, control circuitry 210 may switch to delivery of bipolar energy to continue to modulate the target nerve(s) in the target tissue site at a reduced rate and/or amplitude. Delivery of bipolar energy after the determination of the satisfaction of the threshold condition may allow control circuitry 210 to continue neuromodulation of the target nerve(s) while still reducing energy delivery to non-target tissue.

In some examples, control circuitry 210 may continue to determine values for the one or more parameters as signal generation circuitry 206 delivers bipolar energy to the target tissue site. Control circuitry 210 may determine whether the determined values satisfy a second threshold condition (e.g., a second threshold temperature, a second threshold rate of change/amount of change in temperature, a second threshold impedance, a second threshold rate of change/amount of change in impedance, a second threshold difference between the determined temperature and the average mean temperature, or the like). The second threshold condition is different from a corresponding first threshold condition and can define a higher threshold value. For example, the second threshold temperature may be higher than the first threshold temperature. The second threshold condition may indicate that any additional delivery of energy (e.g., monopolar energy or bipolar energy) to the target tissue site may cause and/or have an increased risk of causing adverse effects to the non-target tissue at the target tissue site. Based on the determination that the second threshold condition is satisfied, control circuitry 210 may instruct signal generation circuitry 206 to terminate delivery of bipolar energy to the target tissue site and/or to power off electrodes 202, thereby terminating all therapeutic energy delivery to the target tissue site. In other examples, based on the determination that the second threshold condition is satisfied, control circuitry 210 may cause communications circuitry 214 to notify the clinician, e.g., to terminate all therapeutic energy delivery to the target tissue site.

In some examples, instead of or in addition to delivering monopolar or bipolar energy to the target tissue site based on the sensed parameters at or near a target tissue site, in some examples, control circuitry 210 is configured to deliver monopolar or bipolar energy to the target tissue site based on a determined distance between target nerve(s) and the vessel wall of the blood vessel.

In some examples, control circuitry 210 is configured to determine, based on sensed parameters at or near the target tissue site (e.g., via sensing circuitry 208) and/or user input (e.g., from the clinician, a distance between a vessel wall of the blood vessel and a target nerve at the target tissue site or a size of the blood vessel (e.g., a blood vessel diameter). Based on a determination that the distance or the size satisfies a threshold condition (e.g., a maximum threshold distance, a minimum threshold distance, a threshold vessel diameter, or the like), control circuitry 210 causes signal generation circuitry 206 to switch from delivering monopolar energy to the target tissue site to delivering bipolar energy to the target tissue site, or vice versa. Switching from delivering monopolar energy to delivering bipolar energy may allow control device 106 to neuromodulate the target nerve(s) at the target tissue site while attempting to reduce/minimize the adverse effects to and/or overheating non-target tissue at the target tissue site. For example, switching to delivering bipolar energy may help reduce temperature of and/or a rate of increase in temperature of non-target tissue at the target tissue site, while still enabling neuromodulation therapy to be delivered to the patient. Thus, the controlled switching from monopolar energy delivery to bipolar energy delivery may enable a neuromodulation procedure to continue on in order to achieve the desired neuromodulation results before the onset of overheating and/or other adverse effects in the non-target tissue.

Control circuitry 210 may automatically determine a type of the blood vessel (e.g., renal vessel, branch vessel, or the like) and/or the blood vessel diameter based on one or more blood flow features sensed by sensing circuitry 208. For example, control circuitry 210 may determine, based on flow separation, that distal portion 108A of elongated member 108 is at a bifurcation in the blood vessel, at a place of tortuosity within the blood vessel, or the like. In some examples, control circuitry 210 determines the diameter of a blood vessel based on impedance values detected by sensing circuitry 208. For example, control circuitry 210 may determine, based on a higher detected impedance, that distal portion 108A is disposed within a blood vessel with a smaller blood vessel diameter, or vice versa. In some examples, control circuitry 210 may receive, via communications circuitry 214, medical imaging of the blood vessel may determine the distance between the vessel wall and the target nerve and/or the blood vessel diameter based on analysis of the medical imaging.

In some examples, control circuitry 210 determines that the distance no longer satisfies the threshold condition and instructs signal generation circuitry 206 to switch from delivering bipolar energy to delivering monopolar energy. Control circuitry 210 may determine, based on a determination that the distance no longer satisfies the threshold condition, that a distance between the target nerve(s) and the vessel wall has increased, such that monopolar energy delivery may be more appropriate to provide efficacious neuromodulation therapy. By instruct signal generation circuitry 206 to switch from bipolar energy delivery to delivering monopolar energy, control circuitry 210 may increase the efficacy of the RF energy by delivering RF energy with increased penetration to the target nerve(s).

In some examples, control circuitry 210 is unable to determine the distance based on, or the on the lack thereof, of the sensed parameters. In some examples, if control circuitry 210 cannot determine the distance, then control circuitry 210 may instruct signal generation circuitry 206 to deliver monopolar energy to the target tissue site. In other examples, if control circuitry 210 cannot determine the distance, then control circuitry 210 may instruct signal generation circuitry 206 to deliver bipolar energy to the target tissue site. Control circuitry 210 may store the determined values, the determined distance, the determined vessel diameter, and/or the threshold conditions in memory 216 or memory of another device.

Communications circuitry 214 supports communication between control device 106 and one or more other computing devices, computing systems, and/or cloud computing environments. Control circuitry 210 of control device 106 may retrieve from the one or more other computing devices, computing systems, and/or cloud computing environments values for one or more threshold conditions, one or more associations between sensed parameters and vessel diameters or distances to target nerves, or the like. Control circuitry 210 may transmit, via communications circuitry 214, the sensed data, the sensed parameters, the determined values, the determined distances, and/or the determined vessel diameters to the one or more other devices, systems, and/or cloud computing environments.

Communications circuitry 214 may communicate with the one or more other devices, systems, and/or cloud computing environments by wireless communication techniques. Wireless communication techniques may include RF communication techniques, e.g., via an antenna (not shown).

UI 212 may be configured to output information to and/or receive input from, e.g., a clinician or another user. The clinician may user UI 212 to input information (e.g., threshold condition values) into control device 106. In some examples, the clinician uses UI 212 to instruct control circuitry 210 and/or signal generation circuitry 206 to begin delivery of RF energy to the target tissue site, terminate delivery of RF energy to the target tissue site (e.g., by powering off electrodes 202), and/or adjust the delivered RF energy to the target tissue site (e.g., by switching between monopolar and bipolar energy, by adjust amplitude of the delivered RF energy). The clinician may interact with UI 212 via one or more inputs including tactile, auditory, and/or visual input.

UI 212 may also output information to the clinician or another user. The outputted information may include, but is not limited, positions of one or more electrodes of electrodes 202 within the patient, the determined values (e.g., temperature and/or impedance values, such as for each of electrodes 202), distances, and/or vessel diameters, the type of RF energy (e.g., monopolar energy and/or bipolar energy) delivered to the patient, or parameters of the RF energy delivered to the patient (e.g., amplitude and/or frequency of the RF energy). In some examples, UI 212 notifies the clinician of a satisfaction of a threshold condition. The notification displayed by UI 212 may include a type of the satisfied threshold condition, a threshold value corresponding to the satisfied threshold condition, and/or a determined value (e.g., determined and/or sensed by control circuitry 210 and/or sensing circuitry 208).

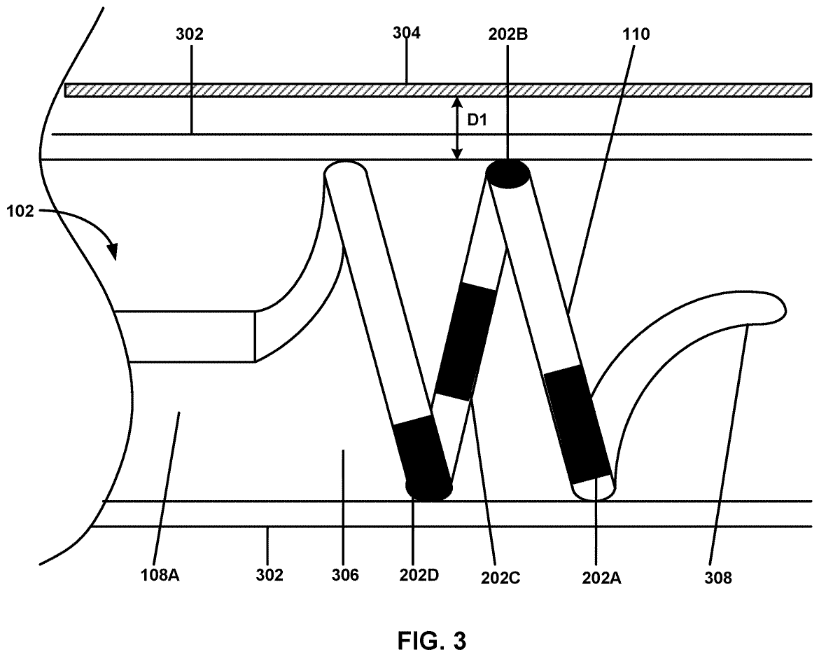

FIG. 3 is a conceptual diagram illustrating distal portion 108A of elongated member 108 of catheter 102 of FIG. 1 positioned within a blood vessel 306. Distal portion 108A of elongated member 108 includes therapy delivery element 110, which includes electrodes 202 disposed along a length of therapy delivery element 110. Therapy delivery element 110 may include a distal tip 308 configured to facilitate navigation of distal portion 108A within vasculature of the patient.

Therapy delivery element 110 may be configured to transform from a relatively low profile delivery configuration (also referred to herein as a collapsed configuration) to a radially expanded configuration within blood vessel 306 (e.g., at a target tissue site). For example, therapy delivery element 110 can include a shape-memory material (e.g., a nitinol wire, a nitinol hollow tubular structure, or the like) that, when disposed within blood vessel 306, is configured to self-expand from the collapsed configuration into the expanded configuration. In some examples, as illustrated in FIG. 3, therapy delivery element 110 may define a spiral or helical shape when therapy delivery element 110 is in the expanded configuration. In other examples, therapy delivery element 110 may define a loop shape, a basket shape, stent-like shape, or other shapes when therapy delivery element 110 is in the expanded configuration. In other examples, therapy delivery element 110 includes a balloon configured to expand form the delivery configuration to the expanded configuration, and one or more electrodes 202 can be positioned on an outer surface of the balloon.

In some examples, system 100 include a guide member (not pictured) configured to facilitate delivery of catheter 102 to a target tissue site within blood vessel 306. The guide member can, for example, be configured to be disposed within or over distal portion 108A and therapy delivery element 110 and hold therapy delivery element 110 in the delivery configuration. The guide member may include but are not limited to, a guide wire, a guide sheath, or the like. A clinician may navigate catheter 102 within the vasculature by advancing elongated member 108 via the guide member (e.g., via the OTW technique, via the RX technique, or the like).

Once therapy delivery element 110 and distal portion 108A are at a target tissue site within blood vessel 306, the clinician may expand therapy delivery element 110 within blood vessel 306, e.g., by proximally retracting the guide member relative to therapy delivery element 110 to a position proximal to therapy delivery element 110. When expanded, therapy delivery element 110 places one or more electrodes of electrodes 202 in apposition with vessel wall 302 of blood vessel 306. When therapy delivery element 110 is in the expanded configuration, control device 106 (not pictured) may deliver RF energy to target nerve(s) 304 via the one or more electrodes of electrodes 202. Target nerve(s) 304 may be separated from an inner surface of vessel wall 302 by a distance D1. Using one or more electrodes 202, signal generation circuitry 206 (FIG. 2) is configured to deliver RF energy (e.g., a monopolar energy and/or a bipolar energy) configured to penetrate vessel wall 302 by at least the distance D1 to modulate target nerve(s) 304, e.g., ablate target nerve(s) 304.

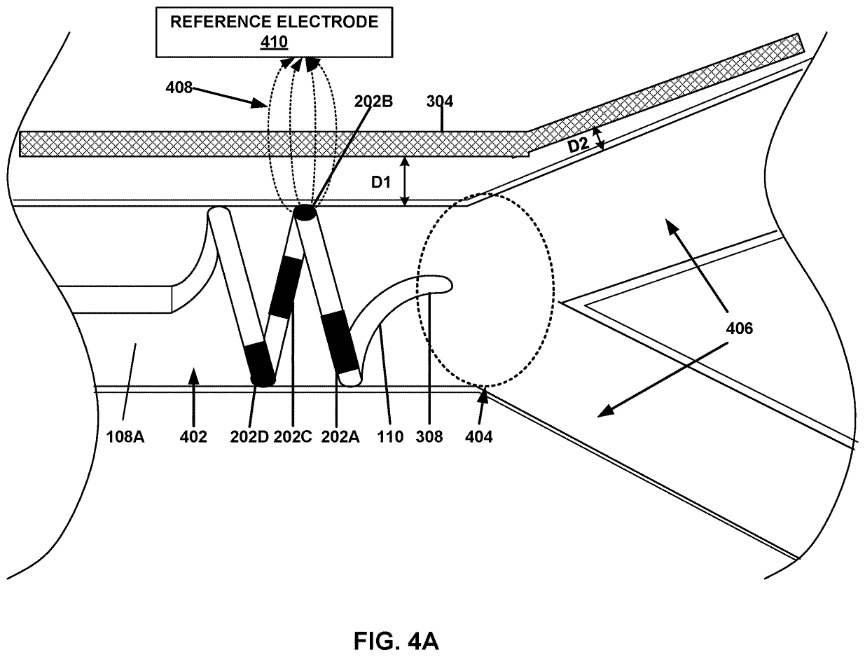

FIG. 4A is a conceptual diagram illustrating an example delivery of monopolar energy 408 via catheter 102 of FIG. 3. As illustrated in FIG. 4A, distal portion 108A and therapy delivery element 110 are disposed within renal vessel 402 (also referred to herein as a main renal vessel 402) and proximal to bifurcation 404 separating renal vessel 402 into branch vessels 406. Target nerve(s) 304 may include one or more renal nerves adjacent to renal vessel 402 and branch vessels 406. In other examples, the devices, systems, and methods described below may be applied to other blood vessels and/or another body lumen. Target nerve(s) 304 is separated from renal vessel 402 by distance D1 and from one or more of branch vessels 406 by a distance D2. In the case of some renal nerves, target nerve(s) 304 are closer to branch vessels 406 and converge distally after a main bifurcation 404. Because target nerve(s) 304 are closer to a blood vessel distally, it may not be necessary to penetrate as deeply into the tissue with energy to modulate (e.g., ablate) target nerve(s) 304. In addition, transmission of the neuromodulation energy transmission may be restricted in some directions by the presence of other arterial branches and veins beyond bifurcation 404. Such anatomical restrictions may obviate the need for deeper penetration of the RF energy and tissue heating.

In some examples, control circuitry 210 determines distance D1 based on one or more sense parameters sensed by sensing circuitry 208 or another sensor (e.g., impedance in tissue surrounding renal vessel 402). This determination of distance D1 can be based on, for example, a proxy parameter, such as a determine diameter of renal vessel 402, or by sensing the target nerve(s) 304 themselves. In some examples, control circuitry 210 determines a diameter of renal vessel 402 and determines distance D1 based on the diameter of renal vessel 402. As an example, control circuitry 210 can correlate the diameter of renal vessel 402 of the patient with distances of target nerve(s) 304 to renal vessel 402. Control circuitry 210 may determine whether distance D1 and/or diameter of renal vessel 402 satisfies a threshold condition (e.g., a maximum threshold distance, a minimum threshold distance, a minimum threshold diameter, a maximum threshold diameter). Based on whether the threshold condition is satisfied, control circuitry 210 may determine a necessary level of penetration of the neuromodulation energy into the tissue to neuromodulate target nerve(s) 304. In some examples, control circuitry 210 determines distance D1, and/or whether the diameter of renal vessel 402 satisfies the threshold condition based on user input or input from another device, such as a medical imaging device.

Based on a determination that at least one threshold condition has been satisfied, control circuitry 210 may determine that distal portion 108A and therapy delivery element 110 are disposed within renal vessel 402 and not within a branch vessel 406. Control device 106 may determine, based on the satisfaction of the at least one threshold condition, that monopolar energy 408 is appropriate for delivering efficacious neuromodulation therapy to target nerve(s) 304, e.g., due to a relatively large distance D1 (compared to D2) between target nerve(s) 304 and the vessel wall of renal vessel 402. Control circuitry 210 can control signal generation circuitry 206 to deliver monopolar energy 408 to target nerve(s) 304 by transmitting an electrical signal from one or more electrodes (e.g., electrode 202B of electrodes 202) of catheter 102 to reference electrode 410.

Reference electrode 410 may include, but are not limited to, one or more electrodes disposed on or within the body of the patient and separate from catheter 102. In some examples, reference electrode 410 includes one or more electrodes disposed on the body of the patient, such as a ground pad. In some examples, reference electrode 410 includes an electrode disposed on a catheter disposed within another blood vessel of the vasculature of the patient. The other blood vessel can include, for example, the aorta or the inferior vena cava (IVC) in some examples in which vessel 402 is a renal artery.

In some examples, multiple reference electrodes 410 may be disposed on and/or within the body of the patient. In such examples, control circuitry 210 may couple each of the one or more electrodes (e.g., electrode 202B) of catheter 102 to one or more reference electrodes 410. Control circuitry 210 may select different combinations of the one or more electrodes and the one or more reference electrodes in accordance with the example methods, devices, and systems described in U.S. Pat. No. 11,304,749, granted on Apr. 19, 2022, and entitled “Systems, devices, and associate methods for neuromodulation with enhanced nerve targeting,” the entire content of which is incorporated herein by reference.

FIG. 4B is a conceptual diagram illustrating an example delivery of bipolar energy 416 via catheter 102 of FIG. 3. As illustrated in FIG. 4B, distal portion 108A and therapy delivery element 110 are disposed one of branch vessels 406 and distal to bifurcation 404 separating renal vessel 402 into branch vessels 406. Branch vessel 406 has a smaller diameter than renal vessel 402. A distal portion 414 of target nerve(s) 304 distal to the bifurcation 404 may be intertwined with branch vessels 406 and may be separated from a vessel wall of one or more of branch vessels 406 by distance D2, which is less than D1 for many patients.

In some examples, control circuitry 210 determines distance D2 based on the one or more parameters sensed by sensing circuitry 208 or another sensor (e.g., impedance in tissue surrounding branch vessel 406). This determination of D2 can be based on, for example, a proxy parameter, such as a determined diameter of branch vessel 406, or by sensing the location of target nerve(s) 304 themselves, e.g., based on tissue impedance specific to the target nerves. In some examples, control circuitry 210 determines a diameter of branch vessel 406 and determines distance D2 based on the diameter of branch vessel 406. As an example, control circuitry 210 can correlate the diameter of branch vessel 406 of the patient with distance of target nerve(s) 304 to branch vessel 406. Control circuitry 210 may determine whether distance D2 and/or the diameter of branch vessel 406 satisfies a threshold condition (e.g., a maximum threshold distance, a minimum threshold distance, a minimum threshold diameter, a maximum threshold diameter). The threshold condition for the distance D2 and/or the diameter of branch vessel 406 may be the same as or less than the threshold condition for distance D1 and/or diameter of branch vessel 406. For example, distance D1 and distance D2 may have a same threshold distance value and control circuitry 210 may determine that a determined distance to target nerve(s) 304 corresponds to distance D1 or to distance D2 based on a determination that the determined distance is greater than or is less than the threshold distance value, respectively. In some examples, control circuitry 210 determined distance D2 and/or whether the diameter of branch vessel 406 satisfies the threshold condition based on user input.

Based on a determination that at least one of the threshold conditions has been satisfied, control circuitry 210 may determine that distal portion 108A and therapy delivery element 110 are disposed within branch vessel 406. Control circuitry 210 may determine, based on the satisfaction of the at least one threshold condition, that efficacious neuromodulation of distal portion 414 may be achieved by bipolar energy 416 and does not require the relatively deeper penetration of monopolar energy 408. For example, because of the relatively smaller distance D2 between the vessel lumen and distal portion 414 of target nerve(s) 304, bipolar energy 416 may be sufficient to neuromodulate distal portion 414 of target nerve(s) 304 without overpenetration of the non-target tissue. Control circuitry 210 can control signal generation circuitry 206 to deliver bipolar energy 416 to distal portion 414 by transmitting an electrical signal from one or more of electrodes 202 (e.g., electrode 202A) to another of electrodes 202 (e.g., electrode 202D) of catheter 102.

In some examples, control circuitry 210 may determine a distance between target nerve(s) 304 and the vessel lumen (e.g., distance D1, distance D2) in accordance with one or more example devices, systems, and methods described in U.S. Pat. No. 10,543,037, issued on Jan. 28, 2020, and entitled “CONTROLLED NEUROMODULATION SYSTEMS AND METHODS OF USE,” the entire content of which is incorporated herein by reference.

While the examples illustrated in FIGS. 4A and 4B illustrated delivery of only monopolar energy 408 or bipolar energy 416 to target nerve(s) 304, in other examples, catheter 102 may deliver monopolar energy 408 and bipolar energy 416 alternatively or simultaneously to target nerve(s) 304. In some examples, a proximal portion of therapy delivery element 110 may be within renal vessel 402 and proximal to bifurcation 404 and a distal portion of therapy delivery element 110 may be within one of branch vessels 406 and distal to bifurcation 404. In such examples, Control circuitry 210 can control signal generation circuitry 206 to deliver monopolar energy 408 to a proximal portion target nerve(s) 304 via the proximal portion of therapy delivery element 110 (e.g., via electrode 202C or electrode 202D) and deliver bipolar energy 416 to distal portion 414 of target nerve(s) 304 via the distal portion of therapy delivery element 110 (e.g., via electrode 202A and electrode 202B).

In other examples, control circuitry 210 may alternate between delivering monopolar energy 408 and bipolar energy 416 to target nerve(s) 304. This can be independent of any sensed parameters or determined vessel sizes in some examples. Alternating delivery of monopolar energy 408 and bipolar energy 416 may increase penetration of RF energy while reducing adverse effects on non-target tissue surround renal vessel 402 and/or branch vessel 406. Control circuitry 210 may switch between monopolar energy 408 and bipolar energy 416 after a predetermined period of time has elapsed, after a number of cycles of either monopolar energy 408 or bipolar energy 416 has been delivered to target nerve(s) 304, or the like.

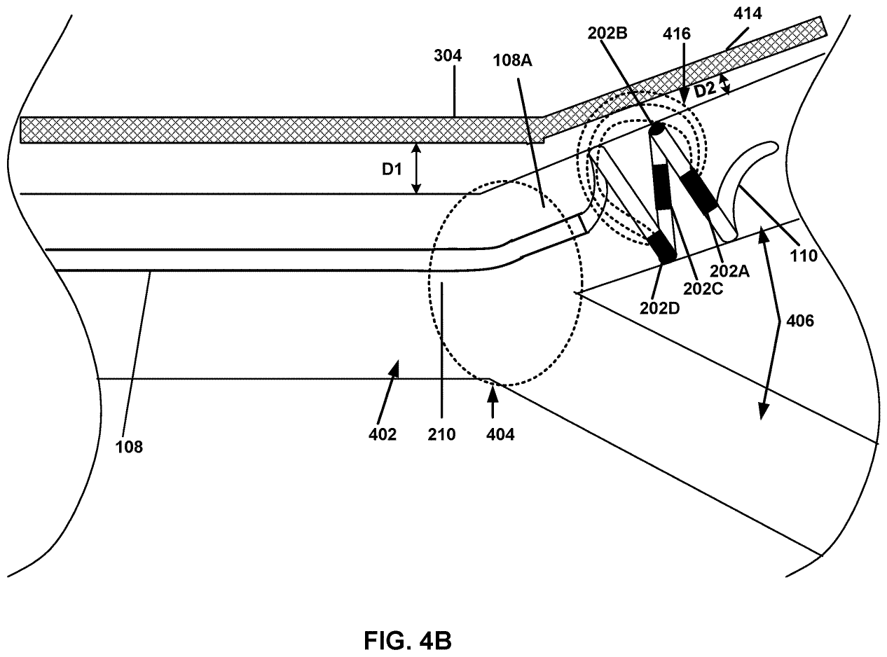

FIG. 5 is a conceptual diagram illustrating example energy delivery profiles of different energy modes of the neuromodulation system of FIG. 1. While FIG. 5 describes energy delivery profiles primarily with respect to RF energy, the example devices, systems, and methods described below may be applied to other electrical stimulation energies (e.g., to microwave energy). FIG. 5 illustrates example energy delivery profiles 502A and 502B (collectively referred to as “energy delivery profiles 502”) for monopolar energy 408 and bipolar energy 416, respectively. Each of energy delivery profiles 502 represents changes in power 504 (e.g., in watts (W)) delivered by the respective energy mode (e.g., monopolar energy 408, bipolar energy 416) over time 506 (e.g., in seconds (s)).

Each of energy delivery profiles 502 include a corresponding initial phase of initial phases 508A, 508B (collectively referred to as “initial phases 508”), a corresponding monitoring phase of monitoring phases 510A, 510B (collectively referred to as “monitoring phases 510”), and a corresponding therapy delivery phase of therapy delivery phases 512A, 512B (collectively referred to as “therapy delivery phases 512”).

Control circuitry 210 may control signal generation circuitry 206 to deliver and adjust RF energy to tissue of the patient based on the corresponding energy delivery profile of energy delivery profiles 502. During initial phase 508, control circuitry 210 may control signal generation circuitry 206 to power on electrodes 202 and begin to deliver RF energy to the tissue of the patient via electrodes 202. Control circuitry 210 may control signal generation circuitry 206 to increase the power of the RF energy as a part of initial phase 508 until the power of the corresponding energy delivery profile reaches a corresponding first threshold power level of first threshold power levels 514A, 514B (collectively referred to as “first threshold power levels 514”). Signal generation circuity 206 may increase the power level of the RF energy linearly, logarithmically, exponentially, or based on one or more other rates of change.

First threshold power levels 514 may be predetermined and stored in memory 216 of control device 106. Corresponding power levels of first threshold power levels 514 may be based on the energy delivery mode (e.g., monopolar energy 408, bipolar energy 416). For example, the power level of first threshold power level 514A of energy delivery profile 502A, corresponding to monopolar energy 408, may be greater than the power level of first threshold power level 514B of energy delivery profile 502B, corresponding to bipolar energy 416.

Once the power level of the RF energy reaches the first threshold power level 514, control circuitry 210 may control signal generation circuitry 206 to incrementally increase power level to a second threshold power level of second threshold power levels 516A-516B (collectively referred to as “second threshold power levels 516”) as a part of monitoring phase 510. Second threshold power level 516 may be predetermined and stored in memory 216 of control device 106. In other examples, control circuitry 210 may determine second threshold power level 516 based on response of the tissue at the target tissue site to the delivered RF energy. The power levels of second threshold power levels 516 may vary based on the corresponding energy delivery mode. For example, second threshold power level 516A may have a higher power level than second threshold power level 516B.

During monitoring phase 510, control circuitry 210 may control signal generation circuitry 206 to incrementally increase the power level of the RF energy. Control circuitry 210 may sense, via sensing circuitry 208, signals and/or parameters (e.g., temperature, impedance) in the tissue in response to the increase in power level determine whether the sensed signals and/or parameters satisfy one or more threshold conditions (e.g., a threshold temperature, a threshold rate of change in temperature, a threshold impedance value, a threshold rate of change in the impedance value). The one or more threshold conditions may correspond to an increased likelihood of overheating and/or adverse effects in the non-target tissue at the target tissue site. Based on a determination that the sensed signals and/or parameters do not satisfy the one or more threshold conditions, control circuitry 210 may control signal generation circuitry 206 to incrementally increase the power level and perform the process until the power level of the RF energy reaches the corresponding second threshold power level of second threshold power levels 516.

During monitoring phase 510, if control circuitry 210 determines that one or more threshold conditions have been satisfied by the sensed signals and/or parameters, then control circuitry 210 may control signal generation circuitry 206 to adjust the corresponding second threshold power level to a maximum power level of the RF energy that does not satisfy the one or more threshold conditions. Control circuitry 210 may then control signal generation circuitry 206 to deliver RF energy during energy delivery phase 512 at a power level less than or equal to the adjusted second threshold power level.

During energy delivery phase 512, control circuitry 210 may control signal generation circuitry 206 to deliver RF energy at a power level less than or equal to the corresponding second threshold power level of second threshold power levels 516 to target nerve(s) 304. Control circuitry 210 may control signal generation circuitry 206 to deliver RF energy at the power level for a predetermined period of time and/or until the sensed parameters satisfy a threshold condition. Once control circuitry 210 determines that the threshold condition is satisfied, control circuitry 210 may control signal generation circuitry 206 to cease delivery of the RF energy to target nerve(s) 304 and power off electrodes 202.

In some examples, for each of the sensed parameters (e.g., temperature, rate of change in temperature, impedance, rate of change in impedance), control circuitry 210 may store a first threshold condition value and a second threshold condition value in memory 216 or receive the threshold conditions from another device. The first threshold condition value may be less than the second threshold condition value. Control circuitry 210 may, based on satisfaction of at least one of the first threshold condition values, control signal generation circuitry 206 to switch from a first energy delivery profile (e.g., energy delivery profile 502A) of energy delivery profiles 502 to a second energy delivery profile (e.g., energy delivery profile 502B). Energy delivery profile 502A and energy delivery profile 502B may correspond to monopolar energy 408 and bipolar energy 416, respectively. Control circuitry 210 may, based on satisfaction of at least one of the second threshold condition values, control signal generation circuitry 206 to cease delivery of all RF energy to target nerve(s) 304 and/or to power off electrodes 202.

When control circuitry 210 controls signal generation circuitry 206 to switch from first energy delivery profile 502A to energy delivery profile 502B, signal generation circuitry 206 may deliver RF energy to target nerve(s) 304 at a corresponding first threshold power level (e.g., first threshold power level 514B) or at a corresponding second threshold power level (e.g., second threshold power level 516B).

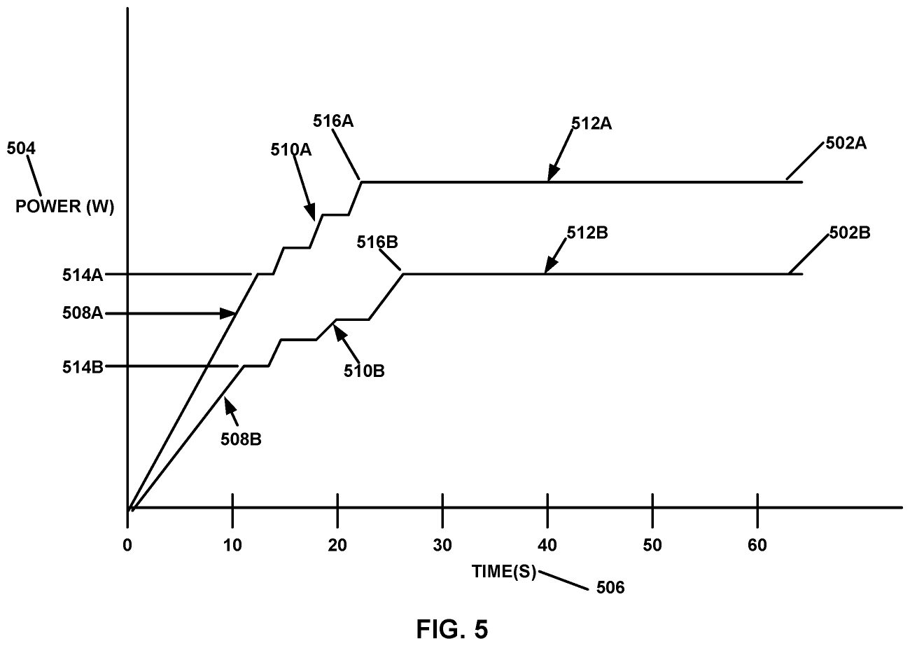

FIG. 6 is a flow diagram illustrating an example method of switching from delivery of monopolar energy (e.g., monopolar energy 408) to delivery of bipolar energy (e.g., bipolar energy 416) based on a position of catheter 102 of FIG. 1 within a patient. While the example method in FIG. 6 is primarily described with regard to neuromodulation of target nerve(s) 304 around a renal vessel of a patient, the example method may be used for neuromodulation of other blood vessel and/or other cavities within the patient. In addition, while the example method in FIG. 6 is primarily described as being performed by control circuitry 210 of control device 106, one or more steps of the example method may be performed by control device 106 based on user input from the clinician.

A clinician may advance elongated member 108 catheter 102 within renal vessel 402 to a target tissue site. The target tissue site may be at, proximal, and/or distal to bifurcation 404 connecting renal vessel 402 to branch vessels 406. The clinician may advance a guide member (e.g., a guidewire) from an incision site on the patient to the target tissue site through vasculature of the patient. The clinician may then advance elongated member 108 along the guide member (e.g., via the OTW technique, via the RX technique) and place distal portion 108A and therapy delivery element 110 at the target tissue site. The clinician may maintain therapy delivery element 110 in a collapsed delivery configuration as the clinician advances therapy delivery element 110 through the vasculature. Once therapy delivery element 110 is at the target tissue site, the clinician may retract the guide member proximally relative to distal portion 108A of catheter 102 to cause therapy delivery element 110 to transform into a radially expanded configuration and place one or more electrodes of electrodes 202 in apposition with the vessel wall at the target tissue site. The clinician may then align the one or more electrodes of electrodes 202 such that target nerve(s) 304 are at least partially positioned between the one or more electrodes and reference electrode 410 disposed on the patient (e.g., on skin of the patient, in another blood vessel or cavity of the patient).

Once distal portion 108A and therapy delivery element 110 are at the target tissue site, control circuitry 210 may control signal generation circuitry 206 to deliver monopolar energy 408 to target nerve(s) 304 at the target tissue site via electrodes 202 on therapy delivery element 110 of catheter 102 (602). Control circuitry 210 may control signal generation circuitry 206 to deliver electric signals from one or more of electrodes 202 to reference electrode 410, e.g., disposed on the patient or otherwise separate from elongated member 108.

Control circuitry 210 may control signal generation circuitry 206 to increase a power level of monopolar energy 408 over time, e.g., in accordance with a corresponding energy delivery profile (e.g., energy delivery profile 502A). Control circuitry 210 may continue to control signal generation circuitry 206 to deliver monopolar energy 408 to target nerve(s) 304 until control circuitry 210 determines that a predetermined treatment period has elapsed and/or signals and/or parameters sensed by sensing circuitry 208 satisfies at least one threshold condition (e.g., a threshold temperature, a threshold rate of change in temperature, a threshold impedance value, a threshold rate of change in the impedance value). Based on the determination, control circuitry 210 may control signal generation circuitry 206 to cease delivery of monopolar energy 408 and/or power off electrodes 202.

Control circuitry 210 receives, from sensing circuitry 208, a signal indicative of a parameter at or near the target tissue site (604). The signal may include one or more electrical signals sensed by sensing circuitry 208 via one or more of electrodes 202. In some examples, the signal may correspond to, but is not limited to, impedance detected by electrodes 202, flow characteristics of blood within the blood vessel, or the like. The parameter may include a distance between the target nerve(s) 304 and an inner surface of vessel wall 302, a blood vessel diameter, or the like.

Control circuitry 210 determines that the sensed parameter satisfies a threshold condition (606). The threshold condition may include, but is not limited to, a threshold distance between target nerve(s) 304 and the inner surface of vessel wall 302, or a threshold diameter of the blood vessel. Control circuitry 210 may determine a distance between target nerve(s) 304 and a vessel wall of a blood vessel (e.g., renal vessel 402, one of branch vessels 406) containing catheter 102 based on impedance values sensed by electrodes 202, based on flow characteristics sensed by electrodes 202, or like. For example, control circuitry 210 may determine the distance between the inner surface of vessel wall 302 to target nerve(s) 304. In some examples, control circuitry 210 may determine the diameter of the blood vessel based on the impedance detected by electrodes 202. For example, control circuitry 210 may correlate a relatively higher detected impedance with a relatively smaller blood vessel diameter, or vice versa. In some examples, control circuitry 210 may determine, based on flow characteristics (e.g., flow separation) detected by electrodes 202, a position of therapy delivery element 110 relative to bifurcation 404 within renal vessel 402. Control circuitry 210 may compared the determined distance and/or the determined blood vessel diameter to the threshold condition to determine, e.g., whether therapy delivery element 110 is within renal vessel 402, at bifurcation 404, or within branch vessel 406.

Based on a satisfaction of the threshold condition, control circuitry 210 controls signal generation circuitry 206 to deliver bipolar energy 416 to target nerve(s) 304 (e.g., to distal portion 414 of target nerve(s) 304) adjacent to the one of branch vessels 406 containing therapy delivery element 110 (608). Control circuitry 210 may select one or more electrodes of electrodes 202 as cathode electrodes and one or more other electrodes of electrode 202 as anode electrodes. Control circuitry 210 may then control signal generation circuitry 206 to deliver electrical energy from the cathode electrodes to the anode electrodes on therapy delivery element 110. Control circuitry 210 may continue to control signal generation circuitry 206 to deliver bipolar energy 416 to target nerve(s) 304 until neuromodulation of target nerve(s) 304 is complete. Control circuitry 210 may then control signal generation circuitry 206 to cease delivery of bipolar energy 416 and/or power off electrodes 202.

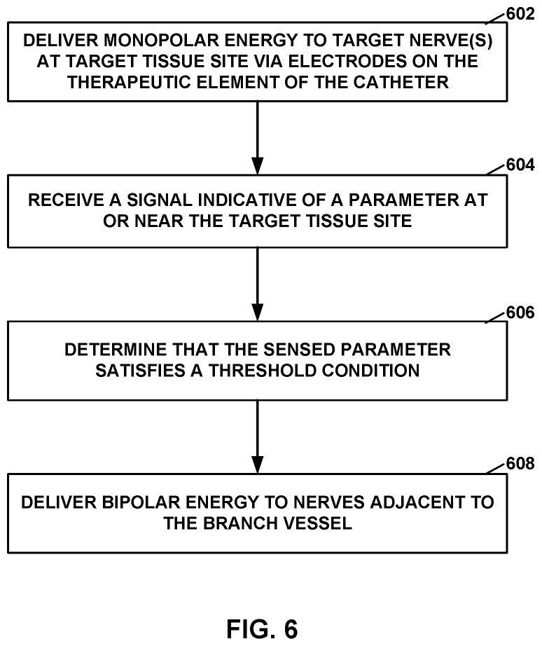

FIG. 7 is a flow diagram illustrating an example method of switching from delivery of monopolar energy 408 to delivery of bipolar energy 416 based on a satisfaction of a threshold condition. While the example method in FIG. 6 is described primarily with regard to neuromodulation of target nerve(s) 304 around a blood vessel (e.g., blood vessel 306) of a patient, the example method may be used for neuromodulation of other cavities within the patient. In addition, while the example method in FIG. 6 is described primarily with regard to steps performed by control circuitry 210 of control device 106 in response to sensed parameters, the example method described in FIG. 7 may be performed by control circuitry 210 in response to user input from a clinician.

Control circuitry 210 controls signal generation circuitry 206 to deliver monopolar energy 408 to target nerve(s) 304 at target tissue site via electrodes 202 on catheter 102 (702). Target tissue site may be a location within blood vessel 306 having target nerve(s) 304 adjacent to vessel wall 302. Control circuitry 210 may, while delivering monopolar energy 408 to target nerve(s) 304, control sensing circuitry 208 to sense parameters from tissue at the target tissue site via electrodes 202 on catheter 102.

Control circuitry 210 determines whether one or more sensed parameters satisfy a first threshold condition (704). The one or more sensed parameters may include temperature around electrodes 202 and/or impedance sensed by electrodes 202. The first threshold condition may include a first threshold value for each of one or more threshold conditions.

The one or more threshold conditions may include, but are not limited to, a threshold temperature, a threshold rate of change/amount of change in temperature over time, a threshold impedance, a threshold rate of change/amount of change in impedance over time, a threshold difference between the determined temperature and a predetermined temperature value (e.g., an average/mean temperature), or the like. Control circuitry 210 may compare the values for the sensed parameters against the first threshold values to determine whether any of the first threshold conditions have been satisfied. Control circuitry 210 may determine whether any of the first threshold conditions have been satisfied by determining whether the sensed parameters is greater than or equal to at least one of the first threshold values.

Based on a determination that the sensed parameters do not satisfy the first threshold condition (“NO” branch of 704), control circuitry 210 controls signal generation circuitry 206 to continue to deliver monopolar energy 408 to target nerve(s) 304 at the target tissue site via electrodes 202 on catheter 102 (702). Based on a determination that the sensed parameters satisfy at least one first threshold condition (“YES” branch of 704), control circuitry 210 controls signal generation circuitry 206 to deliver bipolar energy 416 to target nerve(s) 304 (706). Control circuitry 210 controls device 106 to continue to deliver neuromodulation therapy to the patient while reducing energy delivery to non-target tissue at the target tissue site by controlling signal generation circuitry 206 to switch from delivery monopolar energy 408 to delivery bipolar energy 416. Delivering bipolar energy 416 to the target tissue site may reduce temperature of or a rate of increase in temperature of non-target tissue to prolong neuromodulation of target nerve(s) 304. Furthermore, reducing energy delivery to the non-target tissue may prevent or reduce overheating and/or adverse effects on the non-target tissue. Control circuitry 210 may control sensing circuitry 208 to continue to sense parameters from tissue at the target tissue site via electrodes 202 on catheter 102.

In the example shown in FIG, 7, control circuitry 210 determines whether the sensed parameters satisfy a second threshold condition (708). The second threshold condition may include second threshold values for each of the one or more threshold conditions. The second threshold value may include, is not limited to, a second threshold temperature value, a second threshold rate of change/amount of change in temperature over time, a second threshold impedance value, a second threshold rate of change/amount of change in impedance over time, a second threshold difference between the determined temperature and the predetermined temperature value, or the like. Each of the second threshold value may be greater than or equal to the first threshold value for the same threshold condition. Control circuitry 210 may compare the values for the sensed parameters against the second threshold values to determine whether any of the second threshold conditions have been satisfied.

Based on a determination that the sensed parameters do not satisfy the second threshold condition (“NO” branch of 708), control circuitry 210 controls signal generation circuitry 206 to continue to deliver bipolar energy 416 to target nerve(s) (706). Based on a determination that the sensed parameters satisfy the second threshold condition (“YES” branch of 708), further delivery of neuromodulation therapy to the patient, e.g., at that particular target tissue site, can be ceased. For example, control circuitry 210 can control signal generation circuitry 206 to power off electrodes 202 on catheter 102 (710). Control circuitry 210 controls signal generation circuitry 206 to power off electrodes to cease delivery of any RF energy, monopolar energy 408 or bipolar energy 416, to target nerve(s) 304 and to prevent or reduce overheating of and/or adverse effects on the non-target tissue at the target tissue site.

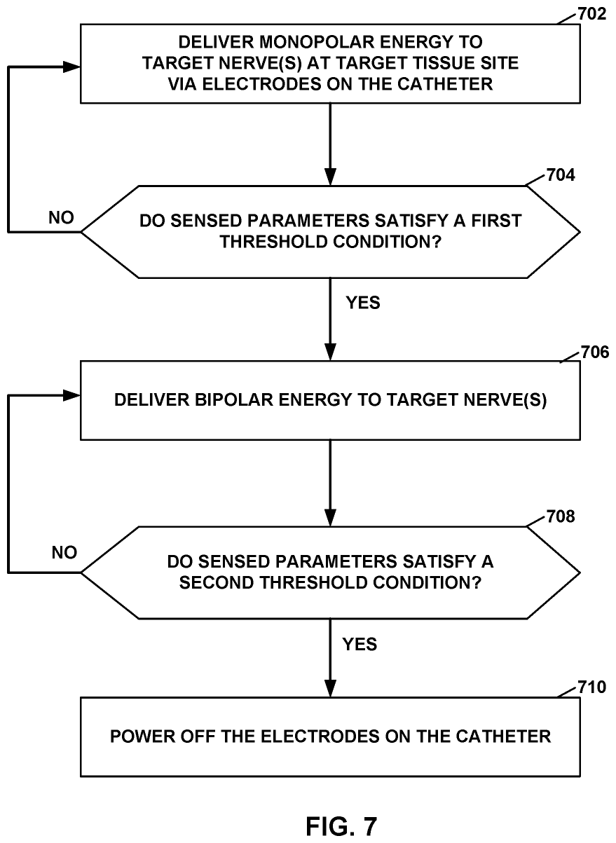

FIG. 8 illustrates an example technique for accessing a renal artery and modulating renal nerves with the system of FIG. 1 in accordance with some examples of the present disclosure. While FIG. 8 illustrates the use of catheter 102 for renal neuromodulation, catheter 102 may be used for other therapies and treatments within another blood vessel or other hollow anatomical body within the human body. Catheter 102 is configured to delivery energy (e.g., RF energy, ultrasound energy, electrical stimulation energy, or the like) to one or more target tissue sites within a renal vessel. Catheter 102 provides access to the renal plexus (RP) through an intravascular path (P), such as a percutaneous access site in the femoral (illustrated), brachial, radial, or axillary artery to the target tissue sites within a respective renal artery (RA). By manipulating proximal portion 108B or elongated member 108 from outside the intravascular path (P), a clinician may advance at least distal portion 108A of elongated member 108 through the sometimes-tortuous intravascular path (P) and remotely manipulate distal portion 108A (FIG. 1) of elongated member 108. Distal portion 108A may be remotely manipulated by the clinician using the handle 104.

In the example illustrated in FIG. 8, distal portion 108A is delivered intravascularly to the treatment site using an inner member 136 in an over-the-wire (OTW) technique. Inner member 136 may be internal to catheter 102 (e.g., a guide wire, inner catheter, or the like) or external to catheter 102 (e.g., an outer sheath or the like). In some examples, inner member 136 is a navigation wire. Catheter 102 may define a passageway for receiving inner member 136 for delivery of catheter 102 using either an OTW or an RX technique. At the treatment site, inner member 136 can be at least partially withdrawn or removed relative to catheter 102 and distal portion 108A can transform into an expanded configuration (e.g., a helical configuration, a spiral configuration, or the like) for delivering ultrasound energy. In other examples, elongated member 108 may be self-steerable such that therapy delivery element 110 may be delivered to the target tissue site without the aid of inner member 136.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced or induced in another suitable manner through the delivery of energy (RF energy, ultrasound energy, microwave energy, or the like). The target tissue site can be within or otherwise proximate to a renal lumen (e.g., a renal artery, a ureter, a renal pelvis, a major renal calyx, a minor renal calyx, or another suitable structure), and the target tissue site can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target tissue devices and associated methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operated through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

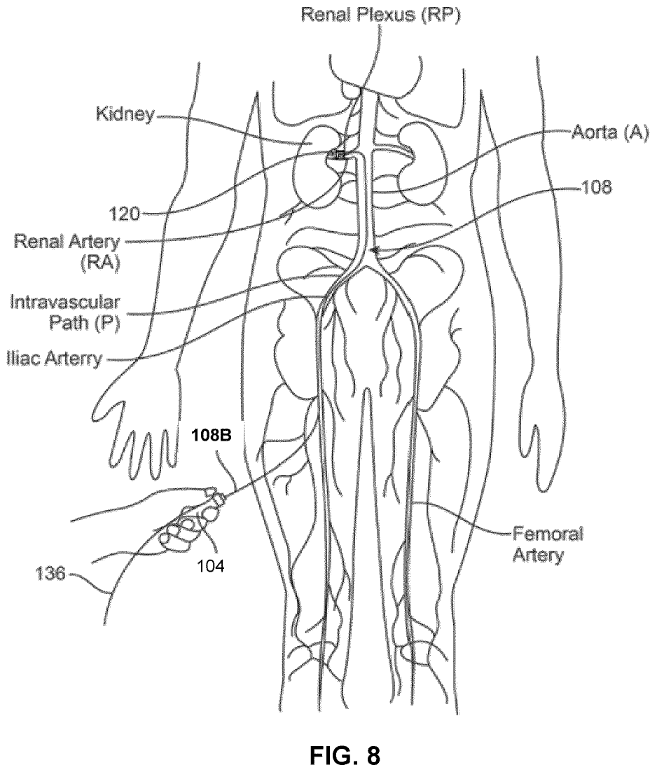

FIG. 9 is a conceptual illustration of an example sympathetic nervous system (SNS) illustrating how the brain communicated with the body via the SNS. As shown in FIG. 9, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

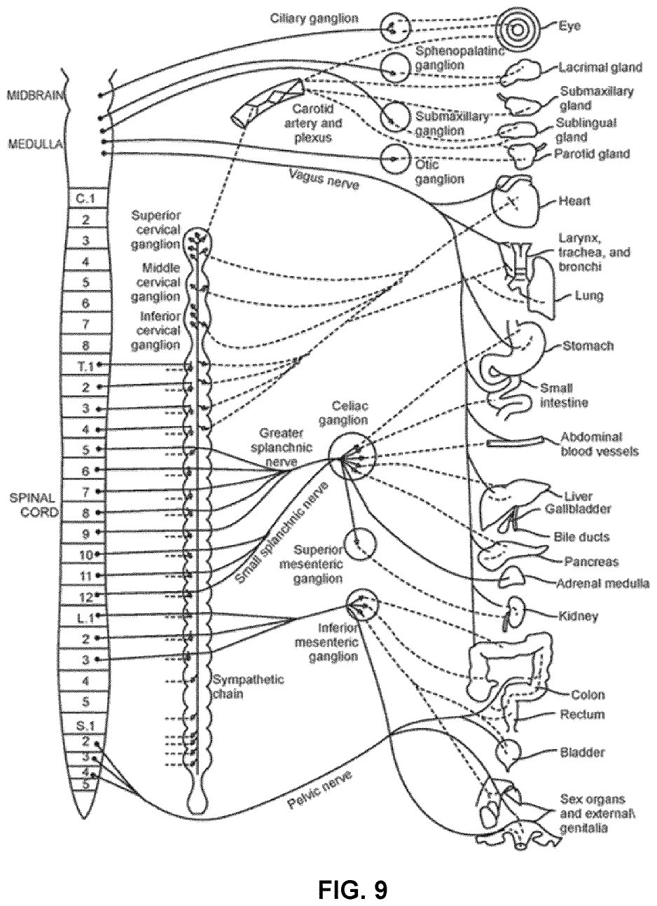

FIG. 10 is an enlarged anatomic view of nerves innervating a left kidney to form the renal plexus surrounding the left renal artery. As FIG. 10 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans.

Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves cause increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication.

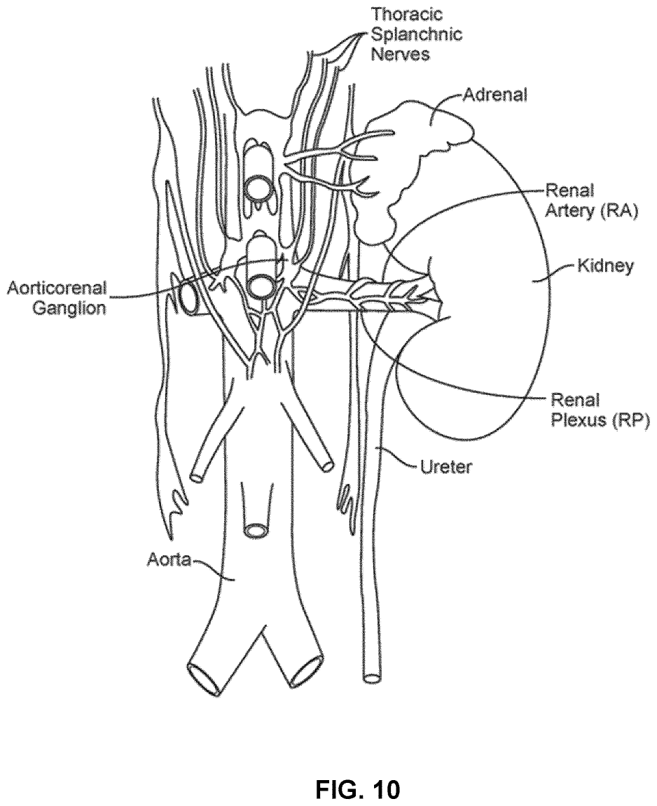

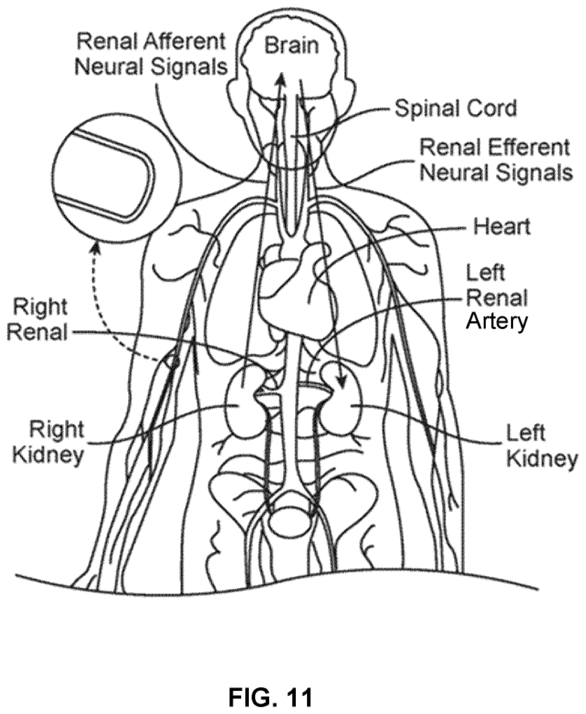

FIG. 11 is an anatomic view of a human body depicting neural efferent and afferent communication between the brain and kidneys. FIG. 12 is a conceptual view of a human body depicting neural efferent and afferent communication between the brain and kidneys. As shown in FIGS. 11 and 12, the afferent communication might be from kidney to the brain or might be from one kidney to the other kidney (via the central nervous system).

These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 11. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

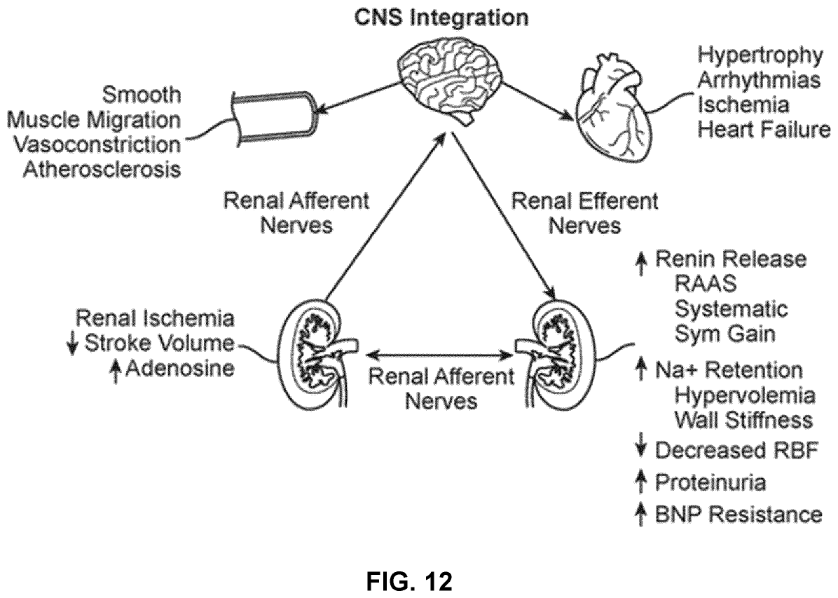

In accordance with the present technology neuromodulation of a left and/or right renal plexus (RP), which is intimately associated with a left and/or right renal artery, may be achieved through intravascular access. FIG. 13 is an anatomic view of the arterial vasculature of a human. As FIG. 13 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

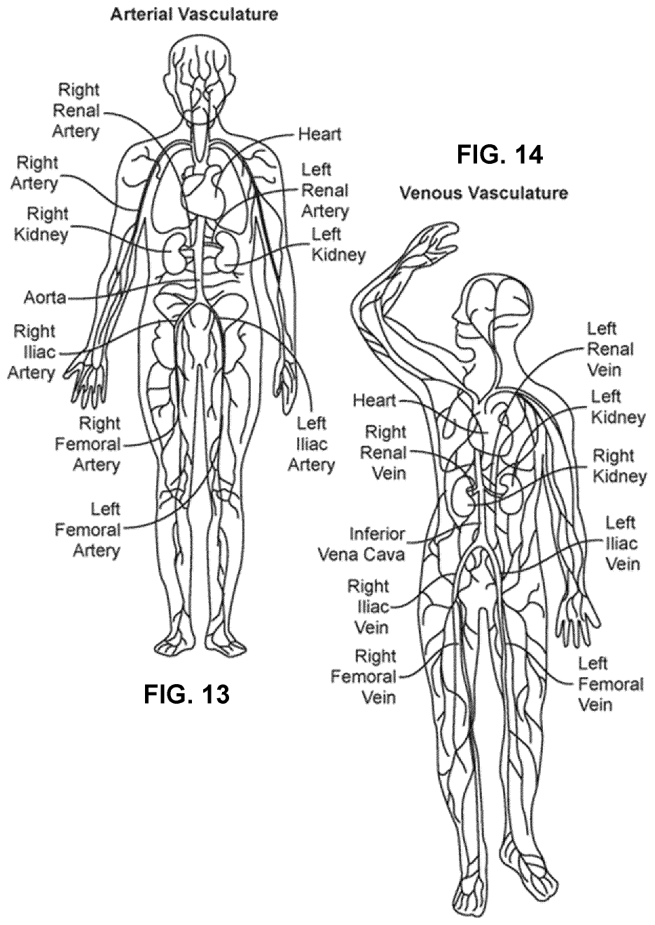

FIG. 14 is an anatomic view of the venous vasculature of a human. As FIG. 14 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base on the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and therapy delivery element 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

The application may be further understood with reference to the following numbered examples.

Example 1: A medical device system comprising: signal generation circuitry configured to deliver energy to a target tissue site within a patient via one or more electrodes of a plurality of electrodes disposed along an elongated body of a catheter; sensing circuitry configured to sense a parameter at or near the target tissue site; and control circuitry configured to: cause the signal generation circuitry to deliver monopolar energy to the target tissue site via one or more electrodes of the plurality of electrodes; determine that the sensed parameter satisfies a threshold condition; and based on determining that the sensed parameter satisfies the threshold condition, cause the signal generation circuitry to deliver bipolar energy to the target tissue site via two or more electrodes of the plurality of electrodes.

Example 2: The medical device system of example 1, wherein the parameter comprises a temperature or a change in temperature over time at or near the target tissue site.

Example 3: The medical device system of example 2, wherein the parameter comprises the temperature, and wherein the threshold condition comprises the temperature being greater than or equal to a threshold temperature value.

Example 4: The medical device system of example 2, wherein the parameter comprises the temperature, and wherein the threshold condition comprises the temperature being less than or equal to a threshold temperature value.

Example 5: The medical device system of example 2, wherein the parameter comprises the temperature, and wherein the threshold condition comprises a difference between the temperature and a stored mean temperature value being greater than or equal to a threshold value.

Example 6: The medical device system of any of examples 1-5, wherein the parameter comprises an impedance of the one or more electrodes of the plurality of electrodes or a change in the impedance over time.

Example 7: The medical device system of example 6, wherein the threshold condition comprises the impedance being greater than or equal to a threshold impedance value.

Example 8: The medical device system of example 6, wherein the threshold condition comprises the impedance being less than or equal to a threshold impedance value.

Example 9: The medical device system of example 2, wherein the parameter comprises the impedance, and wherein the threshold condition comprises a difference between the impedance and a stored mean impedance value being greater than or equal to a threshold value.

Example 10: The medical device system of any of examples 1-8, wherein the threshold condition comprises a first threshold condition, and wherein the control circuitry is further configured to: determine that the sensed parameter satisfies a second threshold condition; and based on a determining that the sensed parameter satisfies the second threshold condition, cause the signal generation circuitry to terminate delivery of the bipolar energy via the two or more electrodes.

Example 11: The medical device system of example 10, wherein the parameter comprises a temperature at or near the target tissue site, and wherein the first threshold condition comprises the temperature being greater than or equal to a first temperature value, and wherein the second threshold condition comprises the temperature being greater than or equal to a second temperature value, the second temperature value being higher than the first temperature value.

Example 12: The medical device system of claim 10, wherein the parameter comprises an impedance of the one or more electrodes of the plurality of electrodes, and wherein the first threshold condition comprises the impedance being greater than or equal to a first impedance value, and wherein the second threshold condition comprises the impedance being greater than or equal to a second impedance value, the second impedance value being higher than the first impedance value.

Example 13: The medical device system of any of examples 1-12, wherein a catheter comprises the plurality of electrodes, the system further comprising a reference electrode separate from the catheter, wherein to deliver the monopolar energy to the target tissue site, the signal generation circuitry is configured to deliver energy via the one or more electrodes and the reference electrode.

Example 14: The medical device system of any of examples 1-13, wherein to deliver the monopolar energy to the target tissue site, the signal generation circuitry is configured to generate a first electrical signal having a first amplitude and to deliver the bipolar energy to the target tissue site, the signal generation circuitry is configured to generate a second electrical signal having a second amplitude, the first amplitude being greater than the second amplitude.

Example 15: The medical device system of any of examples 1-14, wherein to cause the signal generation circuitry to deliver the bipolar energy to the target tissue site, the processing circuity is configured to cause the signal generation circuitry to terminate delivery of the monopolar energy.

Example 16: The medical device system of any of examples 1-15, wherein the signal generation circuitry is further configured to alternate between delivering the monopolar energy and delivering the bipolar energy to the target tissue site.

Example 17: The medical device system of any of examples 1-16, further comprising the catheter, wherein the elongated body is configured to expand from a delivery configuration to an expanded configuration in which the elongated body is configured to place the one or more of the plurality of electrodes in apposition with a wall of a blood vessel.

Example 18: The medical device system of example 17, wherein when in the expanded configuration, the elongated body is configured to define a helix or a spiral.

Example 19: A method comprising: controlling, by control circuitry, signal generation circuitry to deliver monopolar energy via one or more electrodes of a plurality of electrodes of a catheter; receiving, by the control circuitry and from sensing circuitry, a signal indicative of a parameter at or near the target tissue site; determining, by the control circuitry, that the sensed parameter satisfies a threshold condition; and based on determining that the sensed parameter satisfies the threshold condition, controlling, by the control circuitry, the signal generation circuitry to deliver a bipolar energy to the target tissue site via two or more electrodes of the plurality of electrodes.

Example 20: The method of example 19, wherein the parameter comprises a temperature or a change in temperature over time at or near the target tissue site.

Example 21: The method of example 20, wherein the parameter comprises the temperature, and wherein the threshold condition comprises the temperature being greater than or equal to a threshold temperature value.

Example 22: The method of example 20, wherein the parameter comprises the temperature, and wherein the threshold condition comprises the temperature being less than or equal to a threshold temperature value.

Example 23: The method of example 20, wherein the parameter comprises a temperature, and wherein the threshold condition comprises a difference between the temperature and a stored mean temperature value being greater than or equal to a threshold value.

Example 24: The method of any of examples 19-23, wherein the parameter comprises an impedance of the one or more electrodes of the plurality of electrodes or a change in the impedance over time.

Example 25: The method of example 24, wherein the threshold condition comprises the impedance being greater than or equal to a threshold impedance value.

Example 26: The method of example 24, wherein the threshold condition comprises the impedance being less than or equal to a threshold impedance value.

Example 27: The method of example 24, wherein the parameter comprises the impedance, and wherein the threshold condition comprises a difference between the impedance and a stored mean impedance value being greater than or equal to a threshold value.

Example 28: The method of any of examples 19-27, wherein the threshold condition comprises a first threshold condition, and wherein the method further comprises: determining, by the control circuitry, that the sensed parameter satisfies a second threshold condition; and based on determining that the sensed parameter satisfies the second threshold condition, causing, by the control circuitry, cessation of delivery of the monopolar energy and the bipolar energy to the target tissue site.

Example 29: The method of example 28, wherein the parameter comprises a temperature at or near the target tissue site, wherein the first threshold condition comprises the temperature being greater than or equal to a first temperature value, and wherein the second threshold condition comprises the temperature being greater than or equal to a second temperature value, the second temperature value being higher than the first temperature value.

Example 30: The method of example 28, wherein the parameter comprises an impedance of the one or more electrodes of the plurality of electrodes, wherein the first threshold condition comprises the impedance being greater than or equal to a first impedance value, and wherein the second threshold condition comprises the impedance being greater than or equal to a second impedance value, the second impedance value being higher than the first impedance value.

Example 31: The method of any of examples 19-30, wherein a catheter comprises the plurality of electrodes, and wherein delivering the monopolar energy to the target tissue site comprises delivering energy via the one or more electrodes and a reference electrode separate from the catheter.

Example 32: The method of any of examples 19-31, wherein controlling the signal generation circuitry to deliver the monopolar energy comprises controlling, by the control circuitry, the signal generation circuitry to generate a first electrical signal having a first amplitude, and wherein controlling the signal generation circuitry to deliver the bipolar energy comprises controlling, by the control circuitry, the signal generation circuitry to generate a second electrical signal having a second amplitude, the first amplitude being greater than the second amplitude.

Example 33: The method of any of examples 19-32, wherein controlling the signal generation circuitry to deliver the bipolar energy comprises: controlling the signal generation circuitry to terminate delivery of the monopolar energy to the target tissue site.

Example 34: The method of any of examples 19-33, further comprising controlling, by the control circuitry, the signal generation circuitry to alternate between delivering the monopolar energy and delivering the bipolar energy to the target tissue site.

Example 35: A computer-readable medium comprising instructions that, when executed, cause processing circuitry of a computing system to perform the method of any of examples 19-34.

Example 36: A medical device comprising: signal generation circuitry configured to deliver energy to tissue of a patient via a plurality of electrodes of a catheter; sensing circuitry configured to sense a parameter within the patient; and control circuitry configured to: cause signal generation circuitry to deliver monopolar energy to a first target tissue site within a blood vessel of the patient via one or more electrodes of the plurality of electrodes; determine, based on the parameter, a size of the blood vessel at a second target tissue site within the blood vessel or a distance between a nerve and the blood vessel at the second target tissue site; and based on the determined size of the blood vessel or the determined distance, cause the signal generation circuitry to deliver bipolar energy to the second target tissue site via two or more electrodes of the plurality of electrodes.

Example 37: The medical device of example 36, wherein the parameter comprises the distance between the nerve and the blood vessel, and wherein to cause the signal generation circuitry to deliver bipolar energy, the control circuitry is configured to determine the distance is less than or equal to a threshold distance value.

Example 38: The medical device of example 37, wherein the control circuitry is further configured to: determine a position of a nerve relative to the blood vessel; and based on the determined position of the nerve, select the two or more electrodes from the plurality of electrodes for the bipolar energy delivery.

Example 39: The medical device of example 36, wherein the parameter comprises the size of the blood vessel, the size including a diameter of the blood vessel, and wherein the threshold condition comprises the diameter of the blood vessel being less than or equal to a threshold diameter value.

Example 40: The medical device of example 39, wherein the sensing circuitry is configured to sense an impedance of one or more electrodes of the plurality of electrodes, and wherein the control circuitry is configured to determine the diameter of the blood vessel based on the sensed impedance.

Example 41: The medical device of any of examples 36-40, wherein the blood vessel is a renal artery of the patient, wherein the first target tissue site is located proximal to a bifurcation within the renal artery, and wherein the second target tissue site is located distal to the bifurcation.

Example 42: The medical device of example 41, wherein the second target tissue site is located within a branch artery connected to the bifurcation within the renal artery.

Example 43: The medical device of any of examples 41 or 42, wherein the control circuitry is configured to determine that the one or more electrodes are distal to the bifurcation based on the sensed parameter.

Example 44: The medical device of any of examples 36-43, wherein the control circuitry is further configured to cause the signal generation circuitry to switch between delivery of the monopolar energy and delivery of the bipolar energy to one or more of the first target tissue site or the second target tissue site in response to user input.

Example 45: The medical device of any of examples 36-44, wherein a catheter comprises the one or more electrodes, and wherein to deliver the monopolar energy to the first target tissue site, the signal generation circuitry is configured to deliver energy via the one or more electrodes and a reference electrode, the reference electrode being separate from the catheter.

Example 46: The medical device of any of examples 36-45, wherein the control circuitry is further configured to cause the signal generation circuitry to alternate delivery of the monopolar energy and the bipolar energy to one or more of the first target tissue site or the second target tissue site.

Example 47: The medical device of any of examples 36-46, wherein the control circuitry is further configured to cause the signal generation circuitry to simultaneously deliver the monopolar energy to the first target tissue site and the bipolar energy to the second target tissue site.

1. A medical device system comprising:
signal generation circuitry configured to deliver energy to target tissue site within a patient via one or more electrodes of a plurality of electrodes disposed along an elongated body of a catheter;
sensing circuitry configured to sense a parameter at or near the target tissue site; and
control circuitry configured to:
cause the signal generation circuitry to deliver monopolar energy to the target tissue site via one or more electrodes of the plurality of electrodes;
determine that the sensed parameter satisfies a threshold condition; and
based on determining that the sensed parameters satisfies the threshold condition, cause the signal generation circuitry to deliver bipolar energy to the target tissue site via two or more electrodes of the plurality of electrodes.

2. The medical device system of claim 1, wherein the parameter comprises a temperature or a change in temperature over time at or near the target tissue site.

3. The medical device system of claim 2,
wherein the parameter comprises the temperature, and wherein the threshold condition comprises one of:
the temperature being greater than or equal to a threshold temperature value;
the temperature being less than or equal to a threshold temperature value; or
a difference between the temperature and a stored mean temperature value being greater than or equal to a threshold value.

4. The medical device system of claim 1, wherein the parameter comprises an impedance of the one or more electrodes of the plurality of electrodes or a change in the impedance over time.

5. The medical device system of claim 4,
wherein the threshold condition comprises one of:
the impedance being greater than or equal to a threshold impedance value;
the impedance being less than or equal to a threshold impedance value; or
a difference between the impedance and a stored mean impedance value being greater than or equal to a threshold value.

6. The medical device system of claim 1, wherein the threshold condition comprises a first threshold condition, and wherein the control circuitry is further configured to:
determine that the sensed parameter satisfies a second threshold condition; and
based on a determination that the sensed parameter satisfies the second threshold condition, cause the signal generation circuitry to terminate delivery of the bipolar energy via the two or more electrodes.

7. The medical device system of claim 6,
wherein the parameter comprises a temperature at or near the target tissue site, and wherein the first threshold condition comprises the temperature being greater than or equal to a first temperature value, and wherein the second threshold condition comprises the temperature being greater than or equal to a second temperature value, the second temperature value being higher than the first temperature value.

8. The medical device system of claim 1, further comprising:
a catheter, wherein the plurality of electrodes are disposed on the catheter; and
a reference electrode separate from the catheter, wherein to deliver the monopolar energy to the target tissue site, the signal generation circuitry is configured to deliver energy via the one or more electrodes and the reference electrode.

9. The medical device system of claim 1,
wherein to deliver the monopolar energy to the target tissue site, the processing circuitry is configured to cause the signal generation circuitry to generate a first electrical signal having a first amplitude and
wherein to deliver the bipolar energy to the target tissue site, the processing circuitry is configured to cause the signal generation circuitry to generate a second electrical signal having a second amplitude, the first amplitude being greater than the second amplitude.

10. The medical device system of claim 1, wherein to cause the signal generation circuitry to deliver the bipolar energy to the target tissue site, the processing circuitry is configured to cause the signal generation circuitry to terminate delivery of the monopolar energy.

11. The medical device system of claim 1, wherein the signal generation circuitry is further configured to alternate between delivering the monopolar energy and delivering the bipolar energy to the target tissue site.

12-13. (canceled)

14. A method comprising:
controlling, by control circuitry, signal generation circuitry to deliver monopolar energy via one or more electrodes of a plurality of electrodes of a catheter;
receiving, by the control circuitry and from sensing circuitry, a signal indicative of a parameter at or near the target tissue site;
determining, by the control circuitry, that the sensed parameter satisfies a threshold condition; and
based on determining that the sensed parameter satisfies the threshold condition, controlling, by the control circuitry, the signal generation circuitry to deliver a bipolar energy to the target tissue site via two or more electrodes of the plurality of electrodes.

15. The method of claim 14, wherein the parameter comprises a temperature at or near the target tissue site, and wherein the threshold condition comprises one of:
the temperature being greater than or equal to a threshold temperature value;
the temperature being less than or equal to the threshold temperature value; or
a difference the temperature and a stored mean temperature value being greater than or equal to the threshold temperature value.

16. The method of claim 14, wherein the parameter comprises an impedance of the one or more electrodes of the plurality of electrodes, and wherein the threshold condition comprises one of:
the impedance being greater than or equal to a threshold impedance value;
the impedance being less than or equal to the threshold impedance value; or
a difference between the impedance and a stored mean impedance value being greater than or equal to the threshold impedance value.

17. The method of claim 14, wherein the threshold condition comprises a first threshold condition, and wherein the method further comprises:
determining, by the control circuitry, that the sensed parameter satisfies a second threshold condition; and
based on determining that the sensed parameter satisfies the second threshold condition, causing, by the control circuitry, cessation of delivery of the monopolar energy and the bipolar energy to the target tissue site.

18. The method of claim 17, wherein the parameter comprises a temperature at or near the target tissue site, wherein the first threshold condition comprises the temperature being greater than or equal to a first temperature value, and wherein the second threshold condition comprises the temperature being greater than or equal to a second temperature value, the second temperature value being higher than the first temperature value.

19. The method of claim 14, wherein controlling the signal generation circuitry to deliver the monopolar energy comprises controlling, by the control circuitry, the signal generation circuitry to generate a first electrical signal having a first amplitude, and wherein controlling the signal generation circuitry to deliver the bipolar energy comprises controlling, by the control circuitry, the signal generation circuitry to generate a second electrical signal having a second amplitude, the first amplitude being greater than the second amplitude.

20. A medical device comprising
signal generation circuitry configured to deliver energy to tissue of a patient via a plurality of electrodes of a catheter;
sensing circuitry configured to sense a parameter within the patient; and
control circuitry configured to:
cause signal generation circuitry to deliver monopolar energy to a first target tissue site within a blood vessel of the patient via one or more electrodes of the plurality of electrodes;
determine, based on the parameter, a size of the blood vessel at a second target tissue site within the blood vessel or a distance between a nerve and the blood vessel at the second target tissue site; and
based on the determined size of the blood vessel or the determined distance, cause the signal generation circuitry to deliver bipolar energy to the second target tissue site via two or more electrodes of the plurality of electrodes.

21. The medical device of claim 20, wherein the parameter comprises the distance between the nerve and the blood vessel, and wherein to cause the signal generation circuitry to deliver bipolar energy, the control circuitry is configured to determine the distance is less than or equal to a threshold distance value.

22. The medical device of claim 20, wherein the blood vessel is a renal artery of the patient, wherein the first target tissue site is located proximal to a bifurcation within the renal artery, and wherein the second target tissue site is located distal to the bifurcation.
